(19) United States
(12) Patent Application Publication
(10) Pub. No.: US 2023/0346801 A1
(43) Pub. Date: Nov. 2, 2023

(54) USE OF AGENTS FOR TREATMENT OF RESPIRATORY CONDITIONS

(71) Applicant: Sage Therapeutics, Inc., Cambridge, MA (US)

(72) Inventor: Stephen Jay Kanes, Brynlawn, PA (US)

(21) Appl. No.: 17/913,936

(22) PCT Filed: Mar. 24, 2021
(86) PCT No.: PCT/US2021/024010

§ 371 Date: Sep. 23, 2022

Related U.S. Application Data
(60) Provisional application No. 62/994,803, filed on Mar. 25, 2020.
(60) Provisional application No. 62/994,805, filed on Mar. 25, 2020.
(60) Provisional application No. 63/000,415, filed on Mar. 26, 2020.
(60) Provisional application No. 63/000,418, filed on Mar. 26, 2020.
(60) Provisional application No. 63/006,671, filed on Apr. 7, 2020.
(60) Provisional application No. 63/006,672, filed on Apr. 7, 2020.
(60) Provisional application No. 63/063,780, filed on Aug. 10, 2020.
(60) Provisional application No. 63/063,803, filed on Aug. 10, 2020.

(51) Int. Cl. A61K 31/57; A61K 45/06; A61P 11/00

(52) U.S. Cl. CPC A61K 31/57; A61K 45/06; A61P 11/00

(57) ABSTRACT

Provided herein are methods for treating a symptom of a respiratory condition or a disease associated with a coronavirus, in a subject, comprising administering to the subject an effective amount of an agent selected from the group consisting of Compound (1) and Compound (2), or a pharmaceutically acceptable salt thereof.

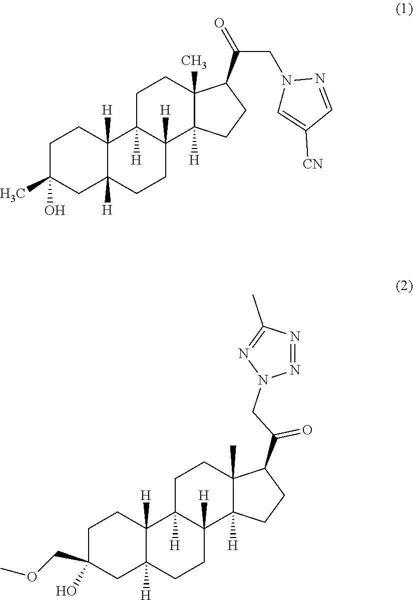

### CROSS-REFERENCE TO RELATED APPLICATIONS

This application claims the benefit of U.S. Provisional Application No. 62/994,805, filed Mar. 25, 2020; U.S. Provisional Application No. 62/994,803, filed Mar. 25, 2020; U.S. Provisional Application No. 63/000,415, filed Mar. 26, 2020; U.S. Provisional Application No. 63/000,418, filed Mar. 26, 2020; U.S. Provisional Application No. 63/006,671, filed Apr. 7, 2020; U.S. Provisional Application No. 63/006,672, filed Apr. 7, 2020; U.S. Provisional Application No. 63/063,780, filed Aug. 10, 2020; and U.S. Provisional Application No. 63/063,803, filed Aug. 10, 2020. These applications are incorporated by reference in their entireties for all purposes.

### TECHNICAL FIELD

The present disclosure relates to methods of treating one or more symptoms of a respiratory condition or a disease associated with a coronavirus by administering an agent as described herein.

### BACKGROUND

SARS-CoV-2 is a coronavirus (CoV) in the family Coronaviridae, subfamily Coronavirinae. These viruses are enveloped viruses with a single-strand, positive-sense RNA genome. Related coronaviruses include severe acute respiratory syndrome coronavirus (SARS-CoV) and Middle East respiratory syndrome coronavirus (MERS-CoV). Compared to SARS-CoV and MERS-CoV, SARS-CoV-2 exhibits a faster human-to-human transmission rate (Huang et al., Lancet 2000, 395, 497), making it particularly challenging to contain and dangerous.

CoVs often originate as enzootic infections that cross the animal-human species barrier and progress to establish zoonotic diseases in humans (Lau et al., PNAS 2005, 102, 14040-5; Rest et al., Infect Genet Evol. 2003, 3, 219-25). Cross-species barrier jumps allowed CoVs such as the SARS CoV and the Middle Eastern respiratory syndrome CoV (MERS) to manifest as virulent human viruses (Schoeman and Fielding, Virology 2019, 16, 69).

The history of creating therapeutics for human coronavirus diseases illustrates the complexity and challenges of the problem.

Accordingly, there is a need for the development of an effective COVID-19 treatment.

### SUMMARY

One aspect of the present invention provides a method of treating a respiratory disease or condition, or a symptom of a respiratory disease or condition in a subject, comprising administering to the subject a therapeutically effective amount of an agent selected from the group consisting of Compound 1

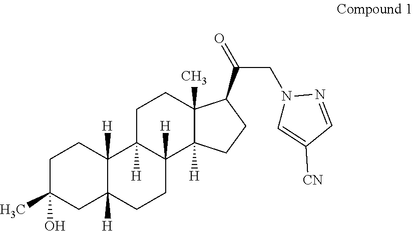

### and Compound 2

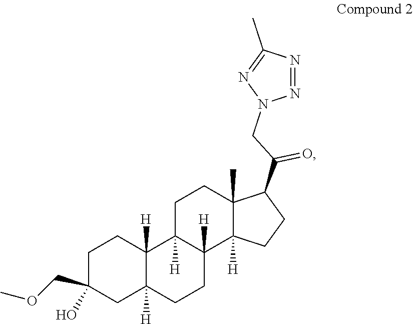

or a pharmaceutically acceptable salt thereof.

Embodiments of this aspect of the invention may include one or more of the following optional features. In some embodiments, the agent is Compound 1. In some embodiments, the agent is a pharmaceutically acceptable salt of Compound 1. In some embodiments, the agent is Compound 2 or a pharmaceutically acceptable salt thereof. In some embodiments, the respiratory condition is respiratory distress. In some embodiments, the respiratory condition is acute respiratory distress syndrome. In some embodiments, the subject exhibits a symptom of the respiratory condition, wherein the symptom is selected from the group consisting of airway hyper-responsiveness, inflammation of lung tissue, lung hypersensitivity, and inflammation-related pulmonary pain. In some embodiments, the inflammation of lung tissue is bronchitis or bronchiectasis. In some embodiments, the inflammation of lung tissue is pneumonia. In some embodiments, the pneumonia is ventilator-associated pneumonia or hospital-acquired pneumonia. In some embodiments, the subject is undergoing or has undergone treatment for an infection, fibrosis, a fibrotic episode, chronic obstructive pulmonary disease, Sarcoidosis (or pulmonary sarcoidosis) or asthma/asthma-related inflammation. In some embodiments, the subject is undergoing or has undergone treatment for an infection. In some embodiments, the infection is a viral infection. In some embodiments, the viral infection is an infection of a virus selected from the group consisting of a coronavirus, an influenza virus, human rhinovirus, a human parainfluenza virus, human metapneumovirus and a hantavirus. In some embodiments, the virus is a coronavirus selected from the group consisting of SARS-CoV, SARS-CoV-2, and MERS-CoV. In some embodiments, the coronavirus is SARS-CoV-2. In some embodiments, the subject has been or is being treated for a disease selected from SARS, COVID-19 or MERS. In some embodiments, the disease is COVID-19. In some embodiments, the infection is a bacterial infection. In some embodiments, the bacterial infection is selected from the group consisting of Streptococcus pneumoniae, Chlamydia pneumoniae, Staphylococcus aureus, Pseudomonas aeruginosa, and Haemophilus influenzae. In some embodiments, the Staphylococcus aureus is methicillin-resistant Staphylococcus aureus. In some embodiments, the subject is undergoing or has undergone treatment for fibrosis or a fibrotic episode. In some embodiments, the fibrosis is cystic fibrosis. In some embodiments, the subject has been previously administered another agent selected from the group consisting of a bronchial muscle/airway relaxant, an antiviral, oxygen, an antibody, and an antibacterial. In some embodiments, the subject has previously been administered the agent described herein and is further administered a second agent selected from a group consisting of a bronchial muscle/airway relaxant, an antiviral, oxygen, and an antibacterial. In some embodiments, the subject has the agent co-administered with a second agent selected from a bronchial muscle/airway relaxant, an antiviral, oxygen, and an antibacterial. In some embodiments, the antiviral is selected from the group consisting of remdesivir, kaletra, lopinavir, and ritonavir. In some embodiments, the antibody is sarilumab or tocilizumab. In some embodiments, the antibacterial is azithromycin. In some embodiments, the subject is being or has been treated with mechanical ventilation. In some embodiments, the subject is being or has been treated with an oxygen mask. In some embodiments, the agent is administered via inhalation, intravenously, by injection (e.g. intramuscularly), subcutaneously, or orally. In some embodiments, the agent is administered intravenously. In some embodiments, the agent is administered orally.

Another aspect of the invention provides a method of treating a respiratory disease or condition, or a symptom of a respiratory disease or condition in a subject undergoing or has undergone treatment for an infection, comprising administering an agent selected from the group consisting of Compound 1 and Compound 2, or a pharmaceutically acceptable salt thereof to the subject.

Embodiments of this aspect of the invention may include one or more of the following optional features. In some embodiments, the infection is an infection of a virus selected from the group consisting of a coronavirus, an influenza virus, human rhinovirus, a human parainfluenza virus, human metapneumovirus and a hantavirus. In some embodiments, the virus a coronavirus selected from the group consisting of SARS-CoV, SARS-CoV-2, and MERS-CoV. In some embodiments, the coronavirus is SARS-CoV-2. In some embodiments, the subject has been or is being treated for a disease selected from SARS, COVID-19 or MERS. In some embodiments, the disease is COVID-19. In some embodiments, the infection is a bacterial infection. In some embodiments, the bacterial infection is selected from the group consisting of Streptococcus pneumoniae, Chlamydia pneumoniae, Staphylococcus aureus, Pseudomonas aeruginosa, and Haemophilus influenzae. In some embodiments, the Staphylococcus aureus is methicillin-resistant Staphylococcus aureus. In some embodiments, the agent is Compound 1. In some embodiments, the agent is a pharmaceutically acceptable salt of Compound 1. In some embodiments, the agent is Compound 2 or a pharmaceutically acceptable salt thereof. In some embodiments, the agent is administered via inhalation, intravenously, by injection (e.g. intramuscularly), subcutaneously, or orally. In some embodiments, the agent is administered intravenously. In some embodiments, the agent is administered orally.

Another aspect of the invention provides a method of treating a respiratory disease or condition, or a symptom of a respiratory disease or condition in a subject comprising administering an agent selected from the group consisting of Compound 1 and Compound 2, or a pharmaceutically acceptable salt thereof to the subject, wherein the subject has been treated with or is being treated with mechanical ventilation.

Embodiments of this aspect of the invention may include one or more of the following optional features. In some embodiments, the subject has acute respiratory distress syndrome. In some embodiments, the agent is Compound 1. In some embodiments, the agent is a pharmaceutically acceptable salt of Compound 1. In some embodiments, the agent is Compound 2 or a pharmaceutically acceptable salt thereof. In some embodiments, the agent is administered via inhalation, intravenously, by injection (e.g. intramuscularly), subcutaneously, or orally. In some embodiments, the agent is administered intravenously. In some embodiments, the agent is administered orally. In some embodiments, the subject is undergoing or has undergone treatment for an infection, comprising administering to the subject an agent selected from the group consisting of Compound 1 and Compound 2, or a pharmaceutically acceptable salt thereof. In some embodiments, the infection is a viral infection comprising an infection of a virus selected from the group consisting of a coronavirus, an influenza virus, human rhinovirus, a human parainfluenza virus, human metapneumovirus and a hantavirus. In some embodiments, the virus is a coronavirus selected from the group consisting of SARS-CoV, SARS-CoV-2, and MERS-CoV. In some embodiments, the coronavirus is SARS-CoV-2. In some embodiments, the subject has been or is being treated for a disease selected from SARS, COVID-19 or MERS. In some embodiments, the disease is COVID-19.

Another aspect of the invention provides a method of treating a respiratory disease or condition, or a symptom of a respiratory disease or condition in a subject comprising administering an agent selected from the group consisting of Compound 1 and Compound 2, or a pharmaceutically acceptable salt thereof to the subject, wherein the subject has been or is being treated for a disease or condition, wherein the disease or condition is selected from the group consisting of cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, pulmonary sarcoidosis, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington, spinocerebellar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, or Sjogren's disease.

Embodiments of this aspect of the invention may include one or more of the following optional features. In some embodiments, the agent is Compound 1. In some embodiments, the agent is a pharmaceutically acceptable salt of Compound 1. In some embodiments, the agent is Compound 2 or a pharmaceutically acceptable salt thereof. In some embodiments, the agent is administered via inhalation, intravenously, by injection (e.g. intramuscularly), subcutaneously, or orally. In some embodiments, the agent is administered intravenously. In some embodiments, the agent is administered orally. In some embodiments, the agent is administered at a rate of 90-160 μg/kg per hour for a therapeutically sufficient duration. In some embodiments, the agent is administered at a rate of 90-150 μg/kg/hour. In some embodiments, the agent is administered at a rate of 130-150 μg/kg/hour. In some embodiments, the agent is administered at a rate of 140-150 μg/kg/hour. In some embodiments, the agent is administered at a rate of about 150 μg/kg/hour. In some embodiments, the agent is administered at a rate of about 140 μg/kg/hour. In some embodiments, the agent is administered at a rate of about 120 μg/kg/hour. In some embodiments, the agent is administered at a rate of about 100 μg/kg/hour. In some embodiments, the agent is administered at a rate of 10-100 μg/kg/hour. In some embodiments, the agent is administered at a rate of 30-80 μg/kg/hour. In some embodiments, the agent is administered at a rate of 35-70 μg/kg/hour. In some embodiments, the agent is administered at a rate of 70 μg/kg/hour. In some embodiments, the agent is administered at a rate of 35 μg/kg/hour. In some embodiments, the agent is administered at a rate of 60-80 μg/kg/hour. In some embodiments, the therapeutically sufficient duration is at least 24 hours. In some embodiments, the therapeutically sufficient duration is at least 48 hours. In some embodiments, the therapeutically sufficient duration is at least 60 hours. In some embodiments, the therapeutically sufficient duration is at least 3 days. In some embodiments, the therapeutically sufficient duration is at least 4 days. In some embodiments, the therapeutically sufficient duration is at least 5 days. In some embodiments, the therapeutically sufficient duration is at least 6 days. In some embodiments, the subject is being treated or has been treated with mechanical ventilation. In some embodiments, the agent is administered to a subject until the subject is no longer being treated with mechanical ventilation.

Another aspect of the invention provides a method of treating a respiratory disease or condition, or a symptom of a respiratory disease or condition in a subject comprising administering an agent selected from the group consisting of Compound 1 and Compound 2, or a pharmaceutically acceptable salt thereof to the subject, wherein the subject has a disease associated with a coronavirus.

Embodiments of this aspect of the invention may include one or more of the following optional features. In some embodiments, the disease is selected from SARS, MERS and COVID-19. In some embodiments, wherein the disease is COVID-19. In some embodiments, the subject suffers from respiratory distress. In some embodiments, the respiratory distress is acute respiratory distress syndrome. In some embodiments, the subject exhibits a symptom selected from the group consisting of airway hyper-responsiveness, inflammation of lung tissue, lung hypersensitivity, and inflammation-related pulmonary pain. In some embodiments, the inflammation of lung tissue is bronchitis or bronchiectasis. In some embodiments, the inflammation of lung tissue is pneumonia. In some embodiments, the pneumonia is ventilator-associated pneumonia or hospital-acquired pneumonia. In some embodiments, the subject is being or has been treated with mechanical ventilation or oxygen treatment. In some embodiments, the subject is being or has been treated with mechanical ventilation. In some embodiments, the agent is Compound 1. In some embodiments, the agent is a pharmaceutically acceptable salt of Compound 1. In some embodiments, the agent is Compound 2 or a pharmaceutically acceptable salt thereof. In some embodiments, the agent is administered via inhalation, intravenously, by injection (e.g. intramuscularly), subcutaneously, or orally. In some embodiments, the agent is administered intravenously. In some embodiments, the agent is administered orally.

In some embodiments, the agent is administered at a rate of 90-160 μg/kg per hour for a therapeutically sufficient duration. In some embodiments, the agent is administered at a rate of 90-150 μg/kg/hour. In some embodiments, the agent is administered at a rate of 130-150 μg/kg/hour. In some embodiments, the agent is administered at a rate of 140-150 μg/kg/hour. In some embodiments, the agent is administered at a rate of about 150 μg/kg/hour. In some embodiments, the agent is administered at a rate of about 140 μg/kg/hour. In some embodiments, the agent is administered at a rate of about 120 μg/kg/hour. In some embodiments, the agent is administered at a rate of about 100 μg/kg/hour. In some embodiments, the agent is administered at a rate of 10-100 μg/kg/hour. In some embodiments, the agent is administered at a rate of 30-80 μg/kg/hour. In some embodiments, the agent is administered at a rate of 35-70 μg/kg/hour. In some embodiments, the agent is administered at a rate of 70 μg/kg/hour. In some embodiments, the agent is administered at a rate of 35 μg/kg/hour. In some embodiments, the agent is administered at a rate of 60-80 μg/kg/hour. In some embodiments, the therapeutically sufficient duration is at least 24 hours. In some embodiments, the therapeutically sufficient duration is at least 48 hours. In some embodiments, the therapeutically sufficient duration is at least 60 hours. In some embodiments, the therapeutically sufficient duration is at least 3 days. In some embodiments, the therapeutically sufficient duration is at least 4 days. In some embodiments, the therapeutically sufficient duration is at least 5 days. In some embodiments, the therapeutically sufficient duration is at least 6 days. In some embodiments, the subject is being treated or has been treated with mechanical ventilation. In some embodiments, the agent is administered to a subject until the subject is no longer being treated with mechanical ventilation. In some embodiments, the patient has acute respiratory distress syndrome. In some embodiments, the patient is intubated. In some embodiments, the patient is under 70 years of age.

In some embodiments, the patient is 70 years of age or older in any one aspect of the present invention.

In one aspect, the invention includes a method of treating a subject with a respiratory condition, comprising administering an agent selected from the group consisting of Compound 1

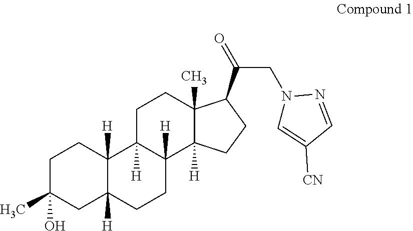

and Compound 2

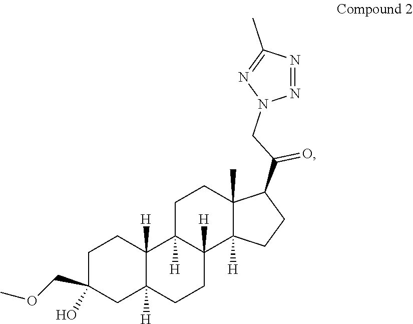

or a pharmaceutically acceptable salt thereof.

In one embodiment of this aspect, the agent is Compound 1.

In another embodiment, the agent is a pharmaceutically acceptable salt of Compound 1.

In one embodiment, the agent is Compound 2 or a pharmaceutically acceptable salt thereof.

In one embodiment, the respiratory condition is respiratory distress.

In a further embodiment, the respiratory condition is acute respiratory distress syndrome.

In one embodiment, the subject exhibits a symptom of the respiratory condition, wherein the symptom is selected from the group consisting of airway hyper-responsiveness, inflammation of lung tissue, lung hypersensitivity, and inflammation-related pulmonary pain.

In another embodiment, the inflammation of lung tissue is bronchitis or bronchiectasis.

In a further embodiment, the inflammation of lung tissue is pneumonia.

In still a further embodiment, the pneumonia is ventilator-associated pneumonia or hospital-acquired pneumonia.

In one embodiment, the subject is undergoing or has undergone treatment for an infection, fibrosis, a fibrotic episode, chronic obstructive pulmonary disease, Sarcoidosis (or pulmonary sarcoidosis) or asthma/asthma-related inflammation.

In one embodiment, the subject is undergoing or has undergone treatment for an infection.

In another embodiment, the infection is a viral infection.

In one embodiment, the viral infection is an infection of a virus selected from the group consisting of a coronavirus, an influenza virus, human rhinovirus, a human parainfluenza virus, human metapneumovirus and a hantavirus.

In a further embodiment, the virus is a coronavirus selected from the group consisting of SARS-CoV, SARS-CoV-2, and MERS-CoV.

In still a further embodiment, the coronavirus is SARS-CoV-2.

In one embodiment, the subject has been or is being treated for a disease selected from SARS, COVID-19 or MERS.

In a further embodiment, the disease is COVID-19.

In one embodiment, the infection is a bacterial infection.

In a further embodiment, the bacterial infection is selected from the group consisting of Streptococcus pneumoniae, Chlamydia pneumoniae, Staphylococcus aureus, Pseudomonas aeruginosa, and Haemophilus influenzae.

In still a further embodiment, the Staphylococcus aureus is methicillin-resistant Staphylococcus aureus.

In one embodiment, the subject is undergoing or has undergone treatment for fibrosis or a fibrotic episode.

In a further embodiment, the fibrosis is cystic fibrosis.

In one embodiment, the subject has been previously administered an agent selected from the group consisting of a bronchial muscle/airway relaxant, an antiviral, oxygen, an antibody, and an antibacterial.

In one embodiment, the subject has previously been administered the agent, further comprising administering to the subject a second agent selected from a group consisting of a bronchial muscle/airway relaxant, an antiviral, oxygen, and an antibacterial.

In one embodiment, the subject has the agent co-administered with an agent selected from a bronchial muscle/airway relaxant, an antiviral, oxygen, and an antibacterial.

In another embodiment, the antiviral is selected from the group consisting of remdesivir, kaletra, lopinavir, and ritonavir.

In another embodiment, the antibody is sarilumab or tocilizumab.

In another embodiment, the antibacterial is azithromycin.

In one embodiment, the subject is being or has been treated with mechanical ventilation.

In one embodiment, the subject is being or has been treated with an oxygen mask.

In one embodiment, the agent is administered via inhalation, intravenously, by injection (e.g. intramuscularly), subcutaneously, or orally.

In a further embodiment, the agent is administered intravenously.

In another embodiment, the agent is administered orally.

In another aspect, the invention includes a method of treating a subject undergoing or has undergone treatment for an infection, comprising administering an agent selected from the group consisting of Compound 1

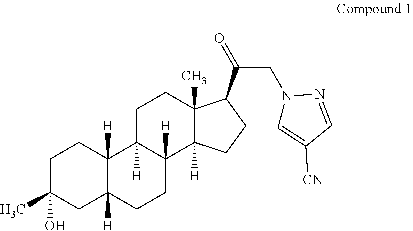

and Compound 2

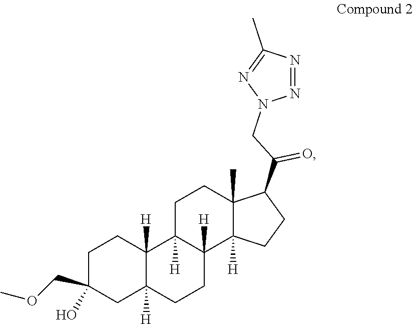

or a pharmaceutically acceptable salt thereof to the subject.

In one embodiment of this aspect, the infection is an infection of a virus selected from the group consisting of a coronavirus, an influenza virus, human rhinovirus, a human parainfluenza virus, human metapneumovirus and a hantavirus.

In another embodiment, the virus is a coronavirus selected from the group consisting of SARS-CoV, SARS-CoV-2, and MERS-CoV.

In a further embodiment, the coronavirus is SARS-CoV-2.

In one embodiment, the subject has been or is being treated for a disease selected from SARS, COVID-19 or MERS.

In a further embodiment, the disease is COVID-19.

In one embodiment, the infection is a bacterial infection.

In a further embodiment, the bacterial infection is selected from the group consisting of Streptococcus pneumoniae, Chlamydia pneumoniae, Staphylococcus aureus, Pseudomonas aeruginosa, and Haemophilus influenzae.

In still a further embodiment, the Staphylococcus aureus is methicillin-resistant Staphylococcus aureus.

In one embodiment, the agent is Compound 1.

In another embodiment, the agent is a pharmaceutically acceptable salt of Compound 1.

In another embodiment, the agent is Compound 2 or a pharmaceutically acceptable salt thereof.

In one embodiment, the agent is administered via inhalation, intravenously, by injection (e.g. intramuscularly), subcutaneously, or orally.

In one embodiment, the agent is administered intravenously.

In another embodiment, the agent is administered orally.

In another aspect, the invention includes a method of treating a subject comprising administering an agent selected from the group consisting of Compound 1

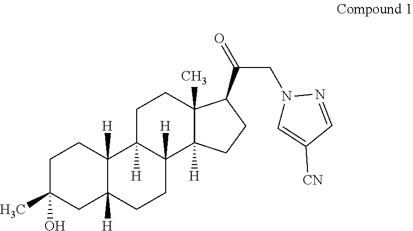

and Compound 2

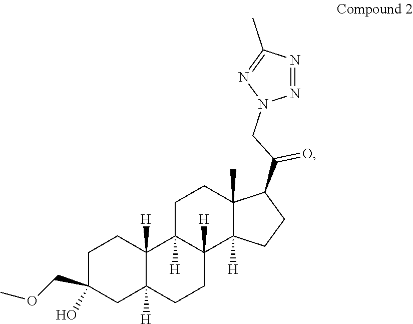

or a pharmaceutically acceptable salt thereof to the subject, wherein the subject has been treated with or is being treated with mechanical ventilation.

In one embodiment, the subject has acute respiratory distress syndrome.

In one embodiment, the agent is Compound 1.

In another embodiment, the agent is a pharmaceutically acceptable salt of Compound 1.

In another embodiment, the agent is Compound 2 or a pharmaceutically acceptable salt thereof.

In one embodiment, the agent is administered via inhalation, intravenously, by injection (e.g. intramuscularly), subcutaneously, or orally.

In one embodiment, the agent is administered intravenously.

In another embodiment, the agent is administered orally.

In one embodiment, the subject is undergoing or has undergone treatment for an infection, the treatment comprising administering to the subject an agent selected from the group consisting of Compound 1 and Compound 2, or a pharmaceutically acceptable salt thereof.

In one embodiment, the infection is a viral infection comprising an infection of a virus selected from the group consisting of a coronavirus, an influenza virus, human rhinovirus, a human parainfluenza virus, human metapneumovirus and a hantavirus.

In one embodiment, the virus is a coronavirus selected from the group consisting of SARS-CoV, SARS-CoV-2, and MERS-CoV.

In a further embodiment, the coronavirus is SARS-CoV-2.

In another embodiment, the subject has been or is being treated for a disease selected from SARS, COVID-19 or MERS.

In a further embodiment, the disease is COVID-19.

In another aspect, the invention includes a method of treating a subject comprising administering an agent selected from the group consisting of Compound 1

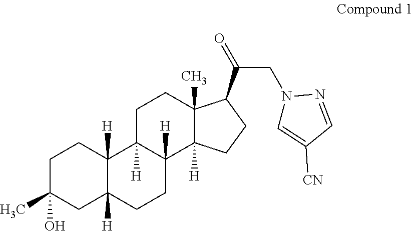

and Compound 2

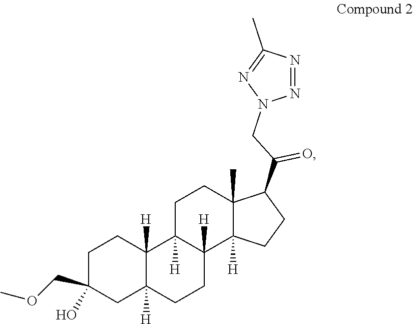

or a pharmaceutically acceptable salt thereof to the subject, wherein the subject has been or is being treated for a disease or condition,
wherein the disease or condition is selected from the group consisting of cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, pulmonary sarcoidosis, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington, spinocerebellar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, or Sjogren's disease.

In one embodiment, the agent is Compound 1.

In another embodiment, the agent is a pharmaceutically acceptable salt of Compound 1.

In another embodiment, the agent is Compound 2 or a pharmaceutically acceptable salt thereof.

In one embodiment, the agent is administered via inhalation, intravenously, by injection (e.g. intramuscularly), subcutaneously, or orally.

In one embodiment, the agent is administered intravenously.

In another embodiment, the agent is administered orally.

In one embodiment, the agent is administered at a rate of 90-160 μg/kg per hour for a therapeutically sufficient duration.

In a further embodiment, the agent is administered at a rate of 90-150 μg/kg/hour.

In a further embodiment, the agent is administered at a rate of 130-150 μg/kg/hour.

In a further embodiment, the agent is administered at a rate of 140-150 μg/kg/hour.

In a further embodiment, the agent is administered at a rate of about 150 μg/kg/hour.

In a further embodiment, the agent is administered at a rate of about 140 μg/kg/hour.

In a further embodiment, the agent is administered at a rate of about 120 μg/kg/hour.

In a further embodiment, the agent is administered at a rate of about 100 μg/kg/hour.

In one embodiment, the agent is administered at a rate of 10-100 μg/kg/hour.

In a further embodiment, the agent is administered at a rate of 30-80 μg/kg/hour.

In a further embodiment, the agent is administered at a rate of 35-70 μg/kg/hour.

In a further embodiment, the agent is administered at a rate of 70 μg/kg/hour.

In a further embodiment, the agent is administered at a rate of 35 μg/kg/hour.

In a further embodiment, the agent is administered at a rate of 60-80 μg/kg/hour.

In one embodiment, the therapeutically sufficient duration is at least 24 hours.

In a further embodiment, the therapeutically sufficient duration is at least 48 hours.

In a further embodiment, the therapeutically sufficient duration is at least 60 hours.

In a further embodiment, the therapeutically sufficient duration is at least 3 days.

In a further embodiment, the therapeutically sufficient duration is at least 4 days.

In a further embodiment, the therapeutically sufficient duration is at least 5 days.

In a further embodiment, the therapeutically sufficient duration is at least 6 days.

In a further embodiment, the subject is being treated or has been treated with mechanical ventilation.

In a further embodiment, the agent is administered to a subject until the subject is no longer being treated with mechanical ventilation.

In another aspect, the invention includes a method of treating a subject comprising administering an agent selected from the group consisting of Compound 1

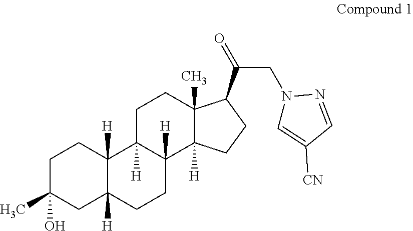

and Compound 2

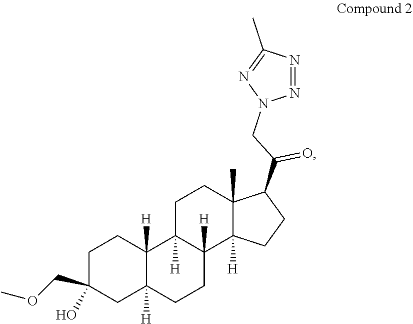

or a pharmaceutically acceptable salt thereof to the subject, wherein the subject has a disease associated with a coronavirus.

In one embodiment, the disease is selected from SARS, MERS and COVID-19.

In a further embodiment, the disease is COVID-19.

In one embodiment, the subject suffers from respiratory distress.

In one embodiment, the respiratory distress is acute respiratory distress syndrome.

In one embodiment, the subject exhibits a symptom selected from the group consisting of airway hyper-responsiveness, inflammation of lung tissue, lung hypersensitivity, and inflammation-related pulmonary pain.

In one embodiment, the inflammation of lung tissue is bronchitis or bronchiectasis.

In another embodiment, the inflammation of lung tissue is pneumonia.

In a further embodiment, the pneumonia is ventilator-associated pneumonia or hospital-acquired pneumonia.

In one embodiment, the subject is being or has been treated with mechanical ventilation or oxygen treatment.

In a further embodiment, the subject is being or has been treated with mechanical ventilation.

In one embodiment, the agent is Compound 1.

In another embodiment, the agent is a pharmaceutically acceptable salt of Compound 1.

In another embodiment, the agent is Compound 2 or a pharmaceutically acceptable salt thereof.

In one embodiment, the agent is administered via inhalation, intravenously, by injection (e.g. intramuscularly), subcutaneously, or orally.

In one embodiment, the agent is administered intravenously.

In another embodiment, the agent is administered orally.

In one embodiment, the agent is administered at a rate of 90-160 μg/kg per hour for a therapeutically sufficient duration.

In a further embodiment, the agent is administered at a rate of 90-150 μg/kg/hour.

In a further embodiment, the agent is administered at a rate of 130-150 μg/kg/hour.

In a further embodiment, the agent is administered at a rate of 140-150 μg/kg/hour.

In a further embodiment, the agent is administered at a rate of about 150 μg/kg/hour.

In a further embodiment, the agent is administered at a rate of about 140 μg/kg/hour.

In a further embodiment, the agent is administered at a rate of about 120 μg/kg/hour.

In a further embodiment, the agent is administered at a rate of about 100 μg/kg/hour.

In one embodiment, the agent is administered at a rate of 10-100 μg/kg/hour.

In a further embodiment, the agent is administered at a rate of 30-80 μg/kg/hour.

In a further embodiment, the agent is administered at a rate of 35-70 μg/kg/hour.

In a further embodiment, the agent is administered at a rate of 70 μg/kg/hour.

In a further embodiment, the agent is administered at a rate of 35 μg/kg/hour.

In a further embodiment, the agent is administered at a rate of 60-80 μg/kg/hour.

In one embodiment, the previous claims, wherein the therapeutically sufficient duration is at least 24 hours.

In a further embodiment, the therapeutically sufficient duration is at least 48 hours.

In a further embodiment, the therapeutically sufficient duration is at least 60 hours.

In a further embodiment, the therapeutically sufficient duration is at least 3 days.

In a further embodiment, the therapeutically sufficient duration is at least 4 days.

In a further embodiment, the therapeutically sufficient duration is at least 5 days.

In a further embodiment, the therapeutically sufficient duration is at least 6 days.

In one embodiment, the subject is being treated or has been treated with mechanical ventilation.

In a further embodiment, the agent is administered to a subject until the subject is no longer being treated with mechanical ventilation.

In one embodiment, the patient has acute respiratory distress syndrome.

In another embodiment, the patient is intubated.

In one embodiment, the patient is under 70 years of age.

In another embodiment, the patient is 70 years of age or older.

In another aspect, the invention includes a method of treating a respiratory disease or condition, or a symptom of a respiratory disease or condition in a subject in a subject, comprising administering to said subject a therapeutically effective amount of a GABA_{A} PAM, or a pharmaceutically acceptable salt or crystalline form thereof, wherein the GABA_{A} PAM is selected from the group consisting of Compound 1

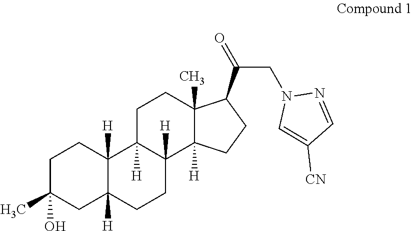

and Compound 2

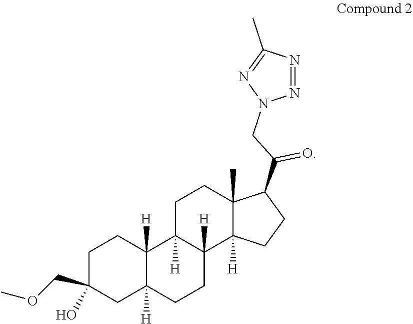

In one embodiment, the respiratory condition is respiratory distress.

In a further embodiment, the respiratory condition is acute respiratory distress syndrome.

In one embodiment, the subject exhibits a symptom of the respiratory condition, wherein the symptom is selected from the group consisting of airway hyper-responsiveness, inflammation of lung tissue, lung hypersensitivity, and inflammation-related pulmonary pain.

In another embodiment, the inflammation of lung tissue is bronchitis or bronchiectasis.

In one embodiment, the inflammation of lung tissue is pneumonia.

In a further embodiment, the pneumonia is ventilator-associated pneumonia or hospital-acquired pneumonia.

In one embodiment, the subject is undergoing or has undergone treatment for an infection, fibrosis, a fibrotic episode, chronic obstructive pulmonary disease, Sarcoidosis (or pulmonary sarcoidosis) or asthma/asthma-related inflammation.

In one embodiment, the subject is undergoing or has undergone treatment for an infection.

In a further embodiment, the infection is a viral infection.

In another embodiment, the viral infection is an infection of a virus selected from the group consisting of a coronavirus, an influenza virus, human rhinovirus, a human parainfluenza virus, human metapneumovirus and a hantavirus.

In a further embodiment, the virus is a coronavirus selected from the group consisting of SARS-CoV, SARS-CoV-2, and MERS-CoV.

In still a further embodiment, the coronavirus is SARS-CoV-2.

In one embodiment, the subject has been or is being treated for a disease selected from SARS, COVID-19 or MERS.

In a further embodiment, the disease is COVID-19.

In one embodiment, the infection is a bacterial infection.

In another embodiment, the bacterial infection is selected from the group consisting of Streptococcus pneumoniae, Chlamydia pneumoniae, Staphylococcus aureus, Pseudomonas aeruginosa, and Haemophilus influenzae.

In a further embodiment, the Staphylococcus aureus is methicillin-resistant Staphylococcus aureus.

In one embodiment, the subject is undergoing or has undergone treatment for fibrosis or a fibrotic episode.

In a further embodiment, the fibrosis is cystic fibrosis.

In one embodiment, the method further comprises administering to the subject one or more additional agents selected from a group consisting of a bronchial muscle/airway relaxant, an antiviral, oxygen, an antibody, and an antibacterial.

In one embodiment, the one or more additional agents are administered to the subject prior to, after, or concurrently with the GABA_{A} PAM.

In one embodiment, the antiviral agent is selected from the group consisting of remdesivir, kaletra, lopinavir, and ritonavir.

In another embodiment, the antibody is sarilumab or tocilizumab.

In another embodiment, the antibacterial is azithromycin.

In one embodiment, the subject is being treated, or has been treated with an oxygen mask.

In another embodiment, the subject is being treated, or has been treated with mechanical ventilation.

In a further embodiment, the GABA_{A} PAM is administered to a subject until the subject is no longer being treated with mechanical ventilation.

In one embodiment, the subject has been or is being treated for a disease or condition selected from the group consisting of cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, pulmonary sarcoidosis, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington, spinocerebellar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, and Sjogren's disease.

In one embodiment, the GABA_{A} PAM is administered via inhalation, intravenously, intramuscularly, subcutaneously, or orally.

In another embodiment, the GABA_{A} PAM is administered orally.

In another embodiment, the GABA_{A} PAM is administered intravenously.

In a further embodiment, the GABA_{A} PAM is administered by continuous intravenous infusion

In one embodiment, the GABA_{A} PAM is administered at a rate of 90-160 μg/kg per hour for a therapeutically effective duration.

In a further embodiment, the GABA_{A} PAM is administered at a rate of 90-150 μg/kg/hour.

In a further embodiment, the GABA_{A} PAM is administered at a rate of 130-150 μg/kg/hour.

In a further embodiment, the GABA_{A} PAM is administered at a rate of 140-150 μg/kg/hour.

In a further embodiment, the GABA_{A} PAM is administered at a rate of about 150 μg/kg/hour.

In a further embodiment, the GABA_{A} PAM is administered at a rate of about 140 μg/kg/hour.

In a further embodiment, the GABA_{A} PAM is administered at a rate of about 120 μg/kg/hour.

In a further embodiment, the GABA_{A} PAM is administered at a rate of about 100 μg/kg/hour.

In another embodiment, the GABA_{A} PAM is administered at a rate of 10-100 μg/kg/hour, for a therapeutically effective duration.

In a further embodiment, the GABA_{A} PAM is administered at a rate of 30-80 μg/kg/hour.

In a further embodiment, the GABA_{A} PAM is administered at a rate of 35-70 μg/kg/hour.

In a further embodiment, the GABA_{A} PAM is administered at a rate of 70 μg/kg/hour.

In a further embodiment, the GABA_{A} PAM is administered at a rate of 35 μg/kg/hour.

In a further embodiment, the GABA_{A} PAM is administered at a rate of 60-80 μg/kg/hour.

In one embodiment, the therapeutically effective duration is at least 24 hours.

In a further embodiment, the therapeutically effective duration is at least 48 hours.

In a further embodiment, the therapeutically effective duration is at least 60 hours.

In a further embodiment, the therapeutically effective duration is at least 3 days.

In a further embodiment, the therapeutically effective duration is at least 4 days.

In a further embodiment, the therapeutically effective duration is at least 5 days.

In a further embodiment, the therapeutically effective duration is at least 6 days.

In one embodiment, the GABA_{A} PAM is administered at a rate of 150 μg/kg/h for about 140 hours.

In one embodiment, the method further comprises the steps of:
a. decreasing the administration rate to about 120 μg/kg/h for one hour;
b. further decreasing the administration rate to about 90 μg/kg/h for one hour;
c. further decreasing the administration rate to about 60 μg/kg/h for one hour; and
d. further decreasing the administration rate to about 30 μg/kg/h for one hour.

In another embodiment, the GABA_{A} PAM is administered at a rate of 70 μg/kg/h for about 58 hours.

In one embodiment, the method further comprises decreasing the rate of administration of the GABA_{A} PAM to about 35 μg/kg/h for about 2 hours.

In one embodiment, treating a respiratory disease or condition in a subject comprises ameliorating one or more symptoms of the respiratory disease or condition.

In another embodiment, the method comprises ameliorating one or more symptoms of acute respiratory distress syndrome in a subject.

In another embodiment, the method comprises ameliorating one or more symptoms of COVID-19 in a subject.

In one embodiment, the symptom is selected from the group consisting of airway hyper-responsiveness, inflammation of lung tissue, lung hypersensitivity, and inflammation-related pulmonary pain.

In another embodiment, the GABA_{A} PAM is administered to the subject in an amount sufficient to increase oxygen saturation in the blood of the subject.

In a further embodiment, the oxygen saturation in the blood of the subject is measured using pulse oximetry.

In one embodiment, the subject is also experiencing a cytokine storm (also known as cytokine release syndrome).

In a further embodiment, the patient has an inflammation resulting from cytokine release syndrome.

In still a further embodiment, the inflammation is a lung inflammation.

In one embodiment, the patient has acute respiratory distress syndrome.

In one embodiment, the patient is intubated.

In one embodiment, the patient is under 70 years of age.

In another embodiment, the patient is 70 years of age or older.

In one embodiment, the GABA_{A} PAM is Compound 1, wherein the Compound 1 is a free base.

In another embodiment, the GABA_{A} PAM is a pharmaceutically acceptable salt of Compound 1.

In one embodiment, the GABA_{A} PAM is Compound 2, wherein the Compound 2 is a free base.

In another embodiment, the GABA_{A} PAM is a pharmaceutically acceptable salt of Compound 2.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DETAILED DESCRIPTION

Described herein are methods of ameliorating one or more symptoms of a respiratory condition in a subject, comprising administering to the subject a therapeutically effective amount of an agent selected from the group consisting of Compound 1

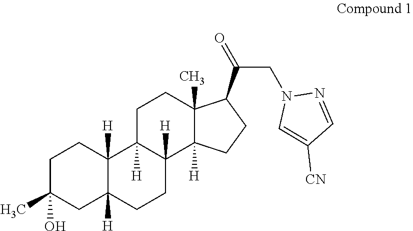

and Compound 2

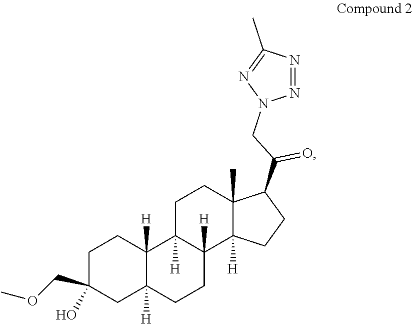

or a pharmaceutically acceptable salt thereof.

Also described herein are methods of ameliorating one or more of a symptom of a disease associated with a coronavirus in a subject, comprising administering to the subject a therapeutically effective amount of an agent selected from the group consisting of Compound 1

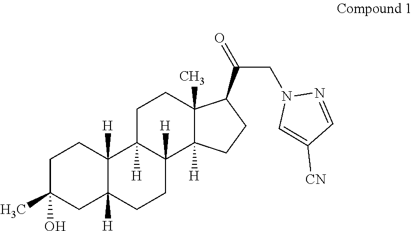

### and Compound 2

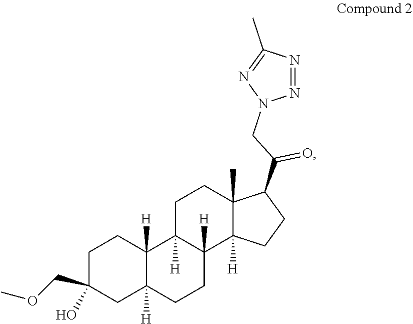

or a pharmaceutically acceptable salt thereof.

### I. DEFINITIONS

As used herein, the term “unit dosage form” refers to the form in which an agent is administered to the subject. Specifically, the unit dosage form can be, for example, a pill, capsule, or tablet.

As used herein, “solid dosage form” means a pharmaceutical dose(s) in solid form, e.g. tablets, capsules, granules, powders, sachets, reconstitutable powders, dry powder inhalers and chewables.

Where the use of the term “about” is before a quantitative value, the present teachings also include the specific quantitative value itself, unless specifically stated otherwise. As used herein, the term “about” refers to a ±10% variation from the nominal value unless otherwise indicated or inferred.

Definitions of specific functional groups and chemical terms are described in more detail below. The chemical elements are identified in accordance with the Periodic Table of the Elements, CAS version, Handbook of Chemistry and Physics, 75^{th} Ed. inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

As used herein, the term “GABA_{A} positive allosteric modulator” refers to a compound that enhances or increases the functional activity of a GABA_{A} receptor.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the present disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, and unless otherwise specified, the terms “treat,” “treating” and “treatment” contemplate an action that occurs while a subject is suffering from the specified disease, disorder or condition, which reduces the severity of disease and/or one or more symptoms of a disease, disorder or condition, or retards or slows the progression of the disease, disorder or condition (“therapeutic treatment”), and also contemplates an action that occurs before a subject begins to suffer from the specified disease, disorder or condition (“prophylactic treatment”).

As used herein, and unless otherwise specified, a “cycle of treatment” comprises administering a first dose of a agent, administering a second dose of the agent, and administering a third dose of the agent, said agent doses being sufficient to treat said subject.

As used herein, and unless otherwise specified, “a therapeutically sufficient duration” comprises a duration of time sufficient to elicit a measurable or observable improvement in a patient's condition upon treatment according to the present disclosure.

In general, the “effective amount” of a compound refers to an amount sufficient to elicit the desired biological response, e.g., to treat symptoms of a respiratory condition. As will be appreciated by those of ordinary skill in this art, the effective amount of a compound of the present disclosure may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

### II. AGENTS

Described herein are methods of treating a subject wherein the subject exhibits one or more symptoms of a respiratory condition and/or has been diagnosed with a respiratory condition, comprising administering to said subject an agent of the present disclosure. In some embodiments an agent is selected from the group consisting of Compound 1

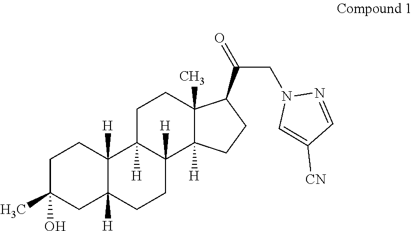

### and Compound 2

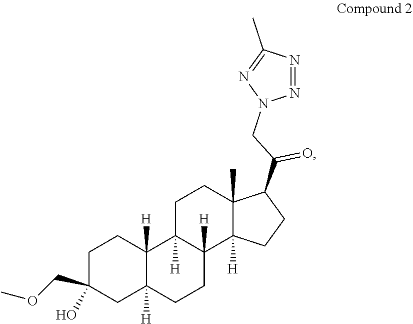

or a pharmaceutically acceptable salt thereof. In some embodiments, the agent is Compound 1. In some embodiments, the agent is Compound 2.

As used herein, “Compound 1” refers to the compound having the formula (or structure):

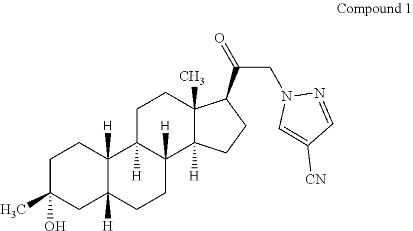

Compound 1 is an agent that has been shown to be a positive allosteric modulator of GABA_{A} receptors (i.e., GABA_{A} PAM) that targets synaptic and extrasynaptic GABA_{A} receptors. As a positive allosteric modulator of GABA_{A} receptors, Compound 1 serves as a therapeutic agent to treat CNS related disorders, e.g., depression, postpartum depression and major depressive disorder. Compound 1 is also known as SAGE-217. In some embodiments, Compound 1 is formulated as zuranolone.

As used herein, “Compound 2” refers to the compound having the formula (or structure):

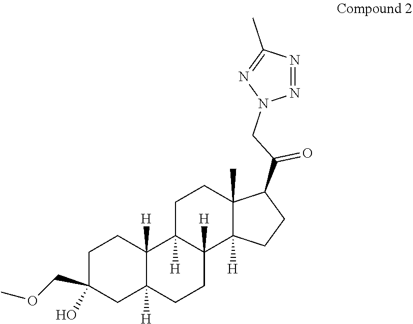

Compound 2 is an agent that has been shown to be a positive allosteric modulator of GABA_{A} receptors (i.e., GABA_{A} PAM) that targets synaptic and extrasynaptic GABA_{A} receptors. As a positive allosteric modulator of GABA_{A} receptors, Compound 2 serves as a therapeutic agent to treat CNS related disorders, e.g., depression, postpartum depression and major depressive disorder.

### III. PHARMACEUTICAL COMPOSITIONS

In one aspect, the disclosure provides a pharmaceutical composition comprising a compound of the present disclosure (also referred to as the “active ingredient”), for example Compound 1 or Compound 2, and a pharmaceutically acceptable excipient. In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, agent is administered to a subject intravenously. In some embodiments, agent is administered to a subject by continuous intravenous infusion.

Generally, the compounds provided herein are administered in an effective amount. The amount of the compound actually administered will typically be determined by a physician, in the light of the relevant circumstances, including the condition to be treated, the chosen route of administration, the actual compound administered, the age, weight, and response of the individual subject, the severity of the subject's symptoms, and the like.

When used to prevent the onset of a disease associated with a coronavirus, the compounds provided herein will be administered to a subject at risk for developing the condition, typically on the advice and under the supervision of a physician, at the dosage levels described above.

The pharmaceutical compositions of the present disclosure may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, a compound is usually a minor component (from about 0.1 to about 50% by weight or from about 1 to about 40% by weight) with the remainder being various vehicles or excipients and processing aids helpful for forming the desired dosing form.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of the present disclosure can also be administered in sustained release forms or from sustained release drug delivery systems. A description of representative sustained release materials can be found in Remington's Pharmaceutical Sciences.

The present disclosure also relates to the pharmaceutically acceptable acid addition salt of a compound of the present disclosure. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

### IV. METHODS OF USE

Described herein are methods of treating a subject wherein the subject exhibits one or more symptoms of a respiratory condition and/or has been diagnosed with a respiratory condition, the method comprising administering to said subject an agent selected from the group consisting of Compound 1

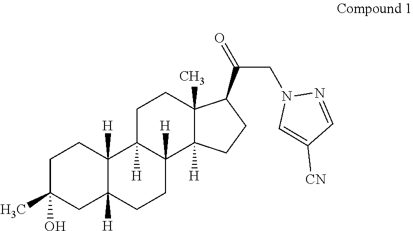

### and Compound 2

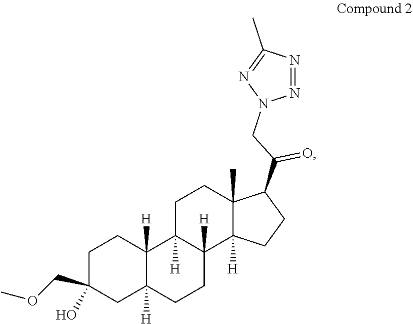

or a pharmaceutically acceptable salt thereof.

In some embodiments, the present disclosure contemplates a method of treating a respiratory disease or condition, or a symptom of a respiratory disease or condition in a subject comprising administering to said subject an agent selected from the group consisting of Compound 1 and Compound 2, or a pharmaceutically acceptable salt thereof, wherein the subject has a respiratory condition. In some embodiments, the agent is Compound 1. In some embodiments, the agent is a pharmaceutically acceptable salt of Compound 1. In some embodiments, Compound is formulated as zuralonone. In some embodiments, the agent is Compound 2 or a pharmaceutically acceptable salt thereof.

In some embodiments, administration of Compound 1, Compound 2, or a pharmaceutically acceptable salt thereof, to a subject exhibiting symptoms of a respiratory condition, may result in the reduction of the severity of one or more symptoms of a respiratory condition or retard or slow the progression of one or more symptoms of a respiratory condition.

In some embodiments, a subject with a respiratory condition has been or is being treated with mechanical ventilation or oxygen (e.g., via an oxygen mask). In some embodiments, a subject with a respiratory condition has been or is being treated with mechanical ventilation. In some embodiments, a subject with a respiratory condition has been or is being treated with oxygen (e.g., via an oxygen mask). In some embodiments, the subject has or has been diagnosed with acute respiratory distress syndrome (ARDS). In some embodiments, the subject exhibits a symptom of the respiratory condition selected from the group consisting of airway hyper-responsiveness, inflammation of lung tissue, lung hypersensitivity, and inflammation-related pulmonary pain.

In one aspect, the present disclosure contemplates a method of treating a respiratory disease or condition, or a symptom of a respiratory disease or condition in a subject (e.g., human subject) that has been or is being treated with mechanical ventilation. In some embodiments, the subject has acute respiratory distress syndrome. In some embodiments, the subject is undergoing or has undergone treatment for an infection, the treatment for an infection comprising administering to the subject an agent.

In one aspect, the present disclosure contemplates a method of treating a respiratory disease or condition, or a symptom of a respiratory disease or condition in a subject (e.g., human subject) having a respiratory condition, comprising administering a first dose, e.g., a load dose, of an agent selected from the group consisting of Compound 1 and Compound 2, or a pharmaceutically acceptable salt thereof, e.g., to a subject under general anesthesia; administering a second dose, e.g., maintenance dose, of the agent, which is lower than said first dose; and administering a third dose, e.g., a downward taper dose, of the agent, said doses being sufficient to treat said subject (e.g., human subject).

In another aspect, the present disclosure contemplates a method of treating a respiratory disease or condition, or a symptom of a respiratory disease or condition in a subject (e.g., human subject) having a respiratory condition, comprising administering a primary dose of an agent selected from the group consisting Compound 1 and Compound 2, or a pharmaceutically acceptable salt thereof, e.g., to a subject under general anesthesia; and administering a subsequent dose of the agent that is lower than the first dose, e.g., a downward taper dose, said doses being sufficient to treat said subject (e.g., human subject).

In some embodiments, the primary dose has one or more of the characteristics of a first dose or load dose as described herein. In some embodiments, the primary dose has one or more of the characteristics of a second dose or maintenance dose as described herein. In some embodiments, the subsequent dose has one or more of the characteristics of a third dose or downward taper dose as described herein.

In some embodiments, the primary dose is administered by continuous IV infusion for at least 24 hours, at least 48 hours, or at least 58 hours. In some embodiments, the primary dose is administered by continuous IV infusion for 24-96 hours, or 48-72 hours, or about 58 hours.

In some embodiments, the subsequent dose is administered after the primary dose, e.g., immediately after. In some embodiments, the primary dose is administered for 0.5-10 hours, or 1-5 hours, or about 2 hours.

In some embodiments, said first dose is a load, e.g., bolus, dose. In some embodiments, said first dose results in a plasma concentration of 50 to 500 nM, 100 to 400 nM, or 200 to 300 nM in a subject. In some embodiments, said first dose results in a plasma concentration of 500 to 1000 nM, 600 to 900 nM, or 700 to 800 nM in a subject. In some embodiments, said first dose results in a plasma concentration of 1000 to 1500 nM, 1100 to 1400 nM, or 1200 to 1300 nM in a subject. In some embodiments, said first dose results in a plasma concentration of 1500 to 2000 nM, 1600 to 1900 nM, or 1700 to 1800 nM in a subject. In some embodiments, said first dose results in a plasma concentration of 2000 to 2500 nM, 2100 to 2400 nM, or 2200 to 2300 nM in a subject. In some embodiments, said first dose results in a plasma concentration of 300 to 800 nM, 400 to 700 nM, or 500 to 600 nM in a subject. In some embodiments, said first dose results in a plasma concentration of 800 to 1300 nM, 900 to 1200 nM, or 1000 to 1100 nM in a subject. In some embodiments, said first dose results in a plasma concentration of 1300 to 1800 nM, 1400 to 1700 nM, or 1500 to 1600 nM in a subject. In some embodiments, said first dose results in a plasma concentration of 1800 to 2300 nM, 1900 to 2200 nM, or 2000 to 2100 nM in a subject. In some embodiments, said first dose results in a plasma concentration of 2300 to 2600 nM, 2400 to 2500 nM in a subject. In some embodiments, said first dose results in a plasma concentration of 300 to 400 nM, 400 to 500 nM, 600 to 700 nM, 800 to 900 nM, 1100 to 1200 nM, 1300 to 1400 nM, 1400 to 1500 nM, 1600 to 1700 nM, 1800 to 1900 nM, 1900 to 2000 nM, 2100 to 2200 nM, 2300 to 2400 nM in a subject. In some embodiments, said first dose results in a plasma concentration of 500 to 2500 nM, 500 to 1500 nM, 500 to 1000 nM, 500 to 800, or 500 to 600 nM in a subject. In some embodiments, said first dose results in a plasma concentration of 50 to 250 nM, 100 to 200 nM, or 140 to 160 nM in a subject. In some embodiments, said first dose results in a plasma concentration of 150±30 nM, 150±20 nM, 150±10 nM, or 150 nM in a subject.

In some embodiments, the plasma concentration of said first dose is measured at a preselected time, e.g., at 10, 15, 20, 30, 45, 60 minutes, 2, 3, 4, 5, 6, 8, 10, 12, 24 hours, 2, 3, 4 days after the initiation of said first dose.

In some embodiments, said first dose is administered over a period of time that is not longer than 6, 5, 4, 3, 2, or 1 hour. In some embodiments, said first dose is administered over a period of time that is at least 10, 20, 30, 40, 50, 60, 70, 80, or 90 minutes in duration. In some embodiments, said first dose is administered over a period of time that is 30 to 120 minutes, 45 to 100 minutes, or 50 to 70 minutes, in duration. In some embodiments, said first dose is administered over a period of time that is 60+/−15 minutes, 60+/−10 minutes, 60+/−5 minutes, or 60 minutes, in duration.

In some embodiments, said first dose is administered at a dosage rate of 200-3500 μg/kg/hour. In some embodiments, said first dose is administered at a dosage rate of 200-350 μg/kg/hour, 250-300 μg/kg/hour, 280-290 μg/kg/hour, 286 μg/kg/hour, 287 μg/kg/hour, or 288 μg/kg/hour, e.g., for one hour.

In some embodiments, said second dose is a maintenance dose. In some embodiments, the administration said second dose is initiated within a preselected time period, wherein said time period begins with the administration of said anesthetic. In some embodiments, the administration said second dose is initiated within a preselected time period, wherein said time period begins with the induction of general anesthesia. In some embodiments, the administration said second dose is initiated within a preselected time period, wherein said time period begins with the beginning of the first dose. In some embodiments, the administration said second dose is initiated within a preselected time period, wherein said time period begins with the end of the first dose. In some embodiments, the administration said second dose is initiated within a preselected time period, wherein said time period begins with the achievement of a predetermined level of the agent, e.g., in the plasma. In some embodiments, said time period begins with the end of the first dose. In some embodiments, said preselected time period begins with beginning or ending of the administration of the first dose and is not longer than 240, 180, 120, 60, 30, 15, or 5 minutes. In some embodiments, said preselected time period begins with beginning or ending of the administration of the first dose and is not longer than 90, 80, 70, or 60 minutes. In some embodiments, the administration of the second dose begins no longer than 90, 80, 70, 60, or 30 minutes after the beginning or end of the administration of the first dose. In some embodiments, the administration of the second dose begins 50 to 70, 55 to 65, or 60 minutes after the beginning or end of the administration of the first dose. In some embodiments, the administration of the second dose begins no more than 60, 50, 40, 30, 20, 10, 5, 4, 3, 2, 1 minute after the end of administration of the first dose. In some embodiments, the administration of the second dose begins at the end of administration of the first dose.

In some embodiments, the administration of the first dose and the initiation of second dose are performed with the same delivery device, e.g., with the same cannula or reservoir.

In some embodiments, said second dose is administered for a period of time that is between 48 and 192 hours, 60 and 144 hours, 60 and 120 hours, 80 and 110 hours, and 90 and 100 hours. In some embodiments, said second dose is administered for 95+/−5 hours. In some embodiments, said second dose is administered for 95 hours. In some embodiments, said second dose is administered for a period of time that is between 24 and 72 hours, 24 and 60 hours, 48 and 72 hours, 48 and 60 hours, or about 58 hours.

In some embodiments, said second dose results in a plasma concentration of 50 to 500 nM, 100 to 400 nM, or 200 to 300 nM in a subject. In some embodiments, said second dose results in a plasma concentration of 500 to 1000 nM, 600 to 900 nM, or 700 to 800 nM in a subject. In some embodiments, said second dose results in a plasma concentration of 1000 to 1500 nM, 1100 to 1400 nM, or 1200 to 1300 nM in a subject. In some embodiments, said second dose results in a plasma concentration of 1500 to 2000 nM, 1600 to 1900 nM, or 1700 to 1800 nM in a subject. In some embodiments, said second dose results in a plasma concentration of 2000 to 2500 nM, 2100 to 2400 nM, or 2200 to 2300 nM in a subject. In some embodiments, said second dose results in a plasma concentration of 300 to 800 nM, 400 to 700 nM, or 500 to 600 nM in a subject. In some embodiments, said second dose results in a plasma concentration of 800 to 1300 nM, 900 to 1200 nM, or 1000 to 1100 nM in a subject. In some embodiments, said first dose results in a plasma concentration of 1300 to 1800 nM, 1400 to 1700 nM, or 1500 to 1600 nM in a subject. In some embodiments, said second dose results in a plasma concentration of 1800 to 2300 nM, 1900 to 2200 nM, or 2000 to 2100 nM in a subject. In some embodiments, said second dose results in a plasma concentration of 2300 to 2600 nM, 2400 to 2500 nM in a subject. In some embodiments, said second dose results in a plasma concentration of 300 to 400 nM, 400 to 500 nM, 600 to 700 nM, 800 to 900 nM, 1100 to 1200 nM, 1300 to 1400 nM, 1400 to 1500 nM, 1600 to 1700 nM, 1800 to 1900 nM, 1900 to 2000 nM, 2100 to 2200 nM, 2300 to 2400 nM in a subject. In some embodiments, said second dose results in a plasma concentration of 500 to 2500 nM, 500 to 1500 nM, 500 to 1000 nM, 500 to 800 nM, or 500 to 600 nM in a subject. In some embodiments, said second dose results in a plasma concentration of 50 to 250 nM, 100 to 200 nM, or 140 to 160 nM in a subject. In some embodiments, said second dose results in a plasma concentration of 150+/−30 nM, 150+/−20 nM, 150+/−10 nM, or 150 nM in a subject.

In some embodiments, plasma concentration of said second dose is measured at a preselected time, e.g., at 10, 15, 20, 30, 45, 60 minutes, 2, 3, 4, 5, 6, 8, 10, 12, 24 hours, 2, 3, 4 days after the initiation of said second dose.

In some embodiments, said second dose results in a plasma concentration of 150 nM, e.g., as measured at a preselected time, e.g., at 10, 15, 20, 30, 45, 60 minutes, 2, 3, 4, 5, 6, 8, 10, 12, 24 hours, 2, 3, 4 days after the initiation of said second dose.

In some embodiments, said second dose is administered at the same infusion rate, e.g. amount of the agent/unit time, over the entire second dose. In some embodiments, the infusion rate, e.g. amount of the agent delivered/unit time varies during the second dose. In some embodiments, said second dose is administered at an infusion rate, e.g. amount of the agent/unit time of 25-1500 μg/kg/hour. In some embodiments, said second dose is administered at an infusion rate, e.g. amount of the agent/unit time of 25-150 μg/kg/hour, 50-100 μg/kg/hour, 75-100 μg/kg/hour, 85 μg/kg/hour, 86 μg/kg/hour, or 87 μg/kg/hour.

In some embodiments, said downward taper dose comprises administering a continuously decreasing amount Compound 1 or Compound 2. In some embodiments, said downward taper dose comprises administering a continuously decreasing amount of Compound 1 or Compound 2 per unit time. In some embodiments, said downward taper dose comprises administering a plurality of step doses, wherein each subsequent step dose is lower than the step dose that precedes it. In some embodiments, said downward taper dose comprises administering a plurality of step doses, wherein each subsequent step dose delivers a lower amount of Compound 1 or Compound 2/unit time than the step dose that precedes it. In some embodiments, said downward taper dose is administered at the same infusion rate, e.g., amount of Compound 1 or Compound 2/unit time, over the entire downward taper dose, wherein the infusion rate of the downward taper dose is less than the infusion rate of the prior dose (e.g., the second dose). For example, the downward taper dose may be administered at about one quarter to about three quarters of the infusion rate of the prior dose, e.g., about one half of the infusion rate of the prior dose. In some embodiments, the downward taper dose is administered for a time period of about one-half hour to about 10 hours, about 1 hour to about 5 hours, or about 2 hours.

In some embodiments, a method comprises administering a first, second, and third step dose. In some embodiments, said first step dose is 60 to 90% of the second/maintenance dose; said second step dose is 40 to 70% of the second/maintenance dose; and said third step dose is 10 to 40% of the second/maintenance dose. In some embodiments, the amount of Compound 1 or Compound 2 delivered/unit time in said first step dose is 60 to 90% of the amount of Compound 1 or Compound 2 delivered/unit time in said second/maintenance dose; the amount of Compound 1 or Compound 2 delivered/unit time in said second step dose is 40 to 70% of the amount of Compound 1 or Compound 2 delivered/unit time in said second/maintenance dose; and the amount of Compound 1 or Compound 2 delivered/unit time in said third step dose is 10 to 40% of the infusion rate, e.g. amount of Compound 1 or Compound 2 delivered/unit time in said second/maintenance dose. In some embodiments, said first step dose is 70 to 80% of the second/maintenance dose; said second step dose is 40 to 60% of the second/maintenance dose; and said third step dose is 20 to 30% of the second/maintenance dose. In some embodiments, the amount of Compound 1 or Compound 2 delivered/unit time in said first step dose is 70 to 80% of the amount of Compound 1 or Compound 2 delivered/unit time in said second/maintenance dose; the amount of Compound 1 or Compound 2 delivered/unit time in said second step dose is 40 to 60% of the amount of Compound 1 or Compound 2 delivered/unit time in said second/maintenance dose; and the amount of Compound 1 or Compound 2 Compound 1 or Compound 2 delivered/unit time in said third step dose is 20 to 30% of the amount of Compound 1 or Compound 2 delivered/unit time in said second/maintenance dose. In some embodiments, said first step dose is 75% of the second/maintenance dose; said second step dose is 50% of the second/maintenance dose; and said third step dose is 25% of the second/maintenance dose. In some embodiments, the amount of Compound 1 or Compound 2 delivered/unit time in said first step dose is 75% of the amount of Compound 1 or Compound 2 delivered/unit time in said second/maintenance dose. In some embodiments, the amount of Compound 1 or Compound 2 delivered/unit time in said second step dose is 50% of the amount of Compound 1 or Compound 2 delivered/unit time in said second/maintenance dose. In some embodiments, the amount of Compound 1 or Compound 2 delivered/unit time in said third step dose is 25% of the amount of Compound 1 or Compound 2 delivered/unit time in said second/maintenance dose.

In some embodiments, after the completion of said third step dose, no Compound 1 or Compound 2 is administered to the subject (e.g., human subject) for at least 10, 20, 30, 40, 50, or 60 days, or until the subject has a subsequent episode of SRSE.

In some embodiments, said first step dose is administered at an amount of agent/unit time of 25-1000 μg/kg/hour. In some embodiments, said first step dose is administered at an amount of agent/unit time of 25-100 μg/kg/hour, 50-75 μg/kg/hour, 60-70 μg/kg/hour, 63 μg/kg/hour, 64 μg/kg/hour, or 65 μg/kg/hour. In some embodiments, said second step dose is administered at an amount of agent/unit time of 10-700 μg/kg/hour. In some embodiments, said second step dose is administered at an amount of agent/unit time of 10-70 μg/kg/hour, 25-55 μg/kg/hour, 40-50 μg/kg/hour, 42 μg/kg/hour, 43 μg/kg/hour, or 44 μg/kg/hour. In some embodiments, said third step dose is administered at an amount of agent/unit time of 5-500 μg/kg/hour. In some embodiments, said third step dose is administered at an amount of agent/unit time of 5-50 μg/kg/hour, 10-35 μg/kg/hour, 15-25 μg/kg/hour, 20 μg/kg/hour, 21 μg/kg/hour, or 22 μg/kg/hour.

Provided herein are methods of administration, for example, of a therapeutic agent (e.g., a agent described herein) or composition comprising a therapeutic agent, to a subject, for example by IV infusion.

In an embodiment, the infusion occurs over at least 1, 2, 3, 4, 5, 6, or 7 days. In an embodiment, the infusion occurs over the course of 1, 2, 3, 4, 5, 6, or 7 days.

In an embodiment, the infusion is bolus infusion (e.g., single dose, single infusion). In an embodiment, the infusion is a plurality of bolus infusions (e.g., multiple bolus infusions, e.g., more than one bolus infusions, e.g., 2, 3, 4, 5 or more bolus infusions). In an embodiment, the plurality of bolus infusions is administered in 1 day, 2 days, 3 days, 4 days, 5 days, 6 days, 1 week, 2 weeks, 3 weeks, 1 month, 2 months, 3 months, 4 months, 5 months, 6 months or more. In an embodiment, the infusion is an intermittent infusion (e.g., an infusion that occurs at irregular intervals). In an embodiment, the infusion is a continuous infusion. In an embodiment, a method comprises administering a plurality of infusions. In an embodiment, a method comprises administering a first, second, and third infusion. In an embodiment, the administration of the second infusion begins no longer than 90, 60, 30, 10, or 5 minutes after the beginning or end of the administration of the first infusion. In an embodiment, the second infusion begins 0 to 90, 0 to 60, 0 to 30, 0 to 10, or 0 to 5 minutes after the beginning or end of the administration of the first infusion. In an embodiment, the second infusion begins no more than 60, 30, 20, 10, 5, 4, 3, 2, or 1 minute(s) after the end of administration of the first infusion. In an embodiment, the second infusion begins at the end of administration of the first infusion. In an embodiment, the first infusion and the initiation of the second infusion are performed with the same delivery device, e.g., with the same cannula or reservoir.

In an embodiment, the amount of agent delivered/unit time varies during the first infusion. In an embodiment, the first (step-up) infusion delivers a smaller amount of agent/unit time than the second (maintenance) infusion. In an embodiment, the first (step-up) infusion comprises administering a plurality of step doses, wherein each subsequent step dose delivers a larger amount of agent/unit time than the step dose that precedes it.

In an embodiment, said third infusion is administered for a period of time that is between 5 and 20 hours, 8 and 16 hours, 10 and 15 hours, or 10 and 13 hours. In an embodiment, said first infusion is administered for 12+/−2 hours. In an embodiment, said first infusion is administered for 12 hours.

In an embodiment, the amount of agent delivered/unit time varies during the first infusion.

In an embodiment, administering said step-up dose comprises administering a continuously increasing amount of agent or a composition comprising an agent as described herein. In an embodiment, administering said step-up dose comprises administering a continuously increasing amount of agent/unit time.

In an embodiment, a method comprises a first, second, and third step dose.

In an embodiment, said first step dose is administered at an amount of agent/unit time of 5-50 μg/kg/hour (e.g., 21.5 μg/kg/hour). In an embodiment, said first step dose is administered at an amount of agent/unit time of 5-50 μg/kg/hour, 10-40 μg/kg/hour, 20-30 μg/kg/hour, 20 μg/kg/hour, 21 μg/kg/hour, 22 μg/kg/hour, or 21.5 μg/kg/hour. In an embodiment, said first step dose is administered at an amount of agent/unit time of 30 μg/kg/hour. In an embodiment, said second step dose is administered at an amount of agent/unit time of 10-100 μg/kg/hour (e.g., 43 μg/kg/hour). In an embodiment, said second step dose is administered at an amount of agent/unit time of 10-100 μg/kg/hour, 20-70 μg/kg/hour, 30-50 μg/kg/hour, 42 μg/kg/hour, 43 μg/kg/hour, or 44 μg/kg/hour. In an embodiment, said second step dose is administered at an amount of agent/unit time of 60 μg/kg/hour. In an embodiment, said third step dose is administered at an amount of agent/unit time of 25-150 μg/kg/hour. In an embodiment, said third step dose is administered at an amount of agent/unit time of 25-150 μg/kg/hour, 40-100 μg/kg/hour, 60-70 μg/kg/hour, 63 μg/kg/hour, 64 μg/kg/hour, 65 μg/kg/hour, or 64.5 μg/kg/hour. In an embodiment, said third step dose is administered at an amount of agent/unit time of 90 μg/kg/hour. In an embodiment, when the agent is agent, a first step dose, second step dose, and third step dose are administered by intermittent infusion, wherein said first step dose is administered at an amount of agent/unit time of 30 μg/kg/hour, said second step dose is administered at an amount of agent/unit time of 60 μg/kg/hour, and said third step dose is administered at an amount of agent/unit time of 90 μg/kg/hour. In an embodiment, when the agent is agent, a first step dose and second step dose are administered by intermittent infusion, wherein said first step dose is administered at an amount of agent/unit time of 30 μg/kg/hour and said second step dose is administered at an amount of agent/unit time of 60 μg/kg/hour.

In an embodiment, the third (step-down/downward taper) infusion delivers a smaller amount of agent/unit time than the second (maintenance) infusion. In an embodiment, the third (step-down/downward taper) infusion comprises administering a plurality of step doses, wherein each subsequent step dose delivers a lower amount of agent/unit time than the step dose that precedes it. In an embodiment, said third infusion is administered for a period of time that is between 5 and 20 hours, 8 and 16 hours, 10 and 15 hours, or 10 and 13 hours. In an embodiment, said third infusion is administered for 12+/−2 hours. In an embodiment, said third infusion is administered for 12 hours.

In an embodiment, administering said downward taper dose comprises administering a continuously decreasing amount of an agent as described herein. In an embodiment, administering said downward taper dose comprises administering a continuously decreasing amount of agent/unit time.

In an embodiment, a method comprises a first, second, and third step dose.

In an embodiment, said first step dose is administered at an amount of agent/unit time of 25-150 μg/kg/hour (e.g., 30 μg/kg/hour). In an embodiment, said first step dose is administered at an amount of agent/unit time of 25-150 μg/kg/hour, 40-100 μg/kg/hour, 60-70 μg/kg/hour, 63 μg/kg/hour, 64 μg/kg/hour, 65 μg/kg/hour, or 64.5 μg/kg/hour. In an embodiment, said second step dose is administered at an amount of agent/unit time of 10-100 μg/kg/hour (e.g., 43 μg/kg/hour). In an embodiment, said second step dose is administered at an amount of agent/unit time of 10-100 μg/kg/hour, 20-70 μg/kg/hour, 30-50 μg/kg/hour, 42 μg/kg/hour, 43 μg/kg/hour, or 44 μg/kg/hour. In an embodiment, said third step dose is administered at an amount of agent/unit time of 5-50 μg/kg/hour (e.g., 21.5 μg/kg/hour). In an embodiment, said third step dose is administered at an amount of agent/unit time of 5-50 μg/kg/hour, 10-40 μg/kg/hour, 20-30 μg/kg/hour, 20 μg/kg/hour, 21 μg/kg/hour, 22 μg/kg/hour, or 21.5 μg/kg/hour.

In an embodiment, a method comprises administering a second/maintenance infusion of 50-150 μg/kg/hour (e.g., 86 μg/kg/hour or 60 μg/kg/hour) of the agent. In an embodiment, the second/maintenance infusion is 50-150 μg/kg/hour, 60-100 μg/kg/hour, 70-90 μg/kg/hour, 85 μg/kg/hour, 86 μg/kg/hour, or 87 μg/kg/hour. In an embodiment, said second/maintenance infusion is administered for a period of time that is between 5 and 80 hours, 10 and 70 hours, 20 and 50 hours, or 30 and 40 hours. In an embodiment, said second/maintenance infusion is administered for 36+/−5 hours. In an embodiment, said second/maintenance infusion is administered for 36 hours. In an embodiment, the plasma concentration of said second/maintenance infusion is measured at a preselected time, e.g., at 10, 15, 20, 30, 45, 60 minutes, 2, 3, 4, 5, 6, 8, 10, 12, 24 hours, 2, 3, 4 days after the initiation of said second/maintenance infusion. In an embodiment, said second/maintenance infusion results in a plasma concentration of 150 nM, e.g., as measured at a preselected time, e.g., at 10, 15, 20, 30, 45, 60 minutes, 2, 3, 4, 5, 6, 8, 10, 12, 24 hours, 2, 3, 4 days after the initiation of said second/maintenance infusion. In an embodiment, said second/maintenance infusion is administered at the same amount of agent/unit time over the entire second/maintenance infusion.

In an embodiment, said first step dose is 10 to 40% (e.g., 25%) of the second/maintenance infusion; said second step dose is 30 to 70% (e.g., 50%) of the second/maintenance infusion; and said third step dose is 60 to 90% (e.g., 75%) of the second/maintenance infusion. In an embodiment, said first step dose is 60 to 90% (e.g., 75%) of the second/maintenance infusion; said second step dose is 30 to 70% (e.g., 50%) of the second/maintenance infusion; and said third step dose is 10 to 40% (e.g., 25%) of the second/maintenance infusion. In an embodiment, the amount of agent delivered/unit time in said first step dose is 10 to 40% (e.g., 25%) of the amount of agent delivered/unit time in said second/maintenance infusion; the amount of agent delivered/unit time in said second step dose is 30 to 70% (e.g., 50%) of the amount of agent delivered/unit time in said second/maintenance infusion; and the amount of agent delivered/unit time in said third step dose is 60 to 90% (e.g., 75%) of the amount of agent delivered/unit time in said second/maintenance infusion. In an embodiment, the amount of agent delivered/unit time in said first step dose is 60 to 90% (e.g., 75%) of the amount of agent delivered/unit time in said second/maintenance infusion; the amount of agent delivered/unit time in said second step dose is 30 to 70% (e.g., 50%) of the amount of agent delivered/unit time in said second/maintenance infusion; and the amount of agent delivered/unit time in said third step dose is 10 to 40% (e.g., 25%) of the amount of agent delivered/unit time in said second/maintenance infusion.

In some embodiments, the agent is administered to a subject at a dosing regimen comprising a continuous intravenous infusion. In some embodiments, the agent is administered to a subject at a dosing regimen over a time period of about 60 hours. In some embodiments, the dosing regimen comprises a continuous intravenous infusion of 70 μg/kg/hour of Compound 1 or Compound 2 from about hour 0 to about hour 58; and 35 μg/kg/hour of Compound 1 or Compound 2 from about hour 58 to about hour 60.

In some embodiments, the agent is administered to a subject at a dosing regimen comprising a continuous intravenous infusion. In some embodiments, the agent is administered to a subject at a dosing regimen over a time period of about 60 hours, wherein the dosing regimen comprises a continuous intravenous infusion of 30 μg/kg/hour of agent from about hour 0 to about hour 4; 60 μg/kg/hour of agent from about hour 4 to about hour 24; 90 μg/kg/hour of agent from about hour 24 to about hour 52; 60 μg/kg/hour of agent from about hour 52 to about hour 56; and 30 μg/kg/hour of agent from about hour 56 to about hour 60.

In some embodiments, an agent is administered to a subject at a dosing regimen comprising a continuous intravenous infusion. In some embodiment, an agent is administered to a subject at a dosing regimen over a time period of about 60 hours, wherein the dosing regimen comprises a continuous intravenous infusion of 30 μg/kg/hour of agent from about hour 0 to about hour 4; 60 μg/kg/hour of agent from about hour 4 to about hour 24; 90 μg/kg/hour of agent from about hour 24 to about hour 52; 60 μg/kg/hour of agent from about hour 52 to about hour 56; and 30 μg/kg/hour of agent from about hour 56 to about hour 60.

In some embodiments, an agent (i.e., GABA_{A} PAM) is administered to a subject at a rate of 10-100 μg/kg per hour for a therapeutically sufficient duration. In some embodiments, an agent is administered to a subject at a rate of 20-100 μg/kg per hour for a therapeutically sufficient duration. In some embodiments, an agent is administered to a subject at a rate of 20-80 μg/kg per hour for a therapeutically sufficient duration. In some embodiments, an agent is administered to a subject at a rate of 30-80 μg/kg per hour for a therapeutically sufficient duration. In some embodiments, an agent is administered to a subject at a rate of 35-70 μg/kg per hour for a therapeutically sufficient duration. In some embodiments, an agent is administered to a subject at a rate of 50-100 μg/kg per hour for a therapeutically sufficient duration. In some embodiments, an agent is administered to a subject at a rate of 60-80 μg/kg per hour for a therapeutically sufficient duration. In some embodiments, an agent is administered to a subject at a rate of 65-75 μg/kg per hour for a therapeutically sufficient duration. In some embodiments, an agent is administered to a subject at a rate of 70 μg/kg per hour for a therapeutically sufficient duration. In some embodiments, an agent is administered to a subject at a rate of 35 μg/kg per hour for a therapeutically sufficient duration.

In some embodiments, an agent is administered to a subject at a rate of 90-160 μg/kg per hour for a therapeutically sufficient duration. In some embodiments, an agent is administered to a subject at a rate of 90-150 μg/kg/hour for a therapeutically sufficient duration. In some embodiments, an agent is administered to a subject at a rate of 110-150 μg/kg/hour for a therapeutically sufficient duration. In some embodiments, an agent is administered to a subject at a rate of 120-150 μg/kg/hour for a therapeutically sufficient duration. In some embodiments, an agent is administered to a subject at a rate of 130-150 μg/kg/hour for a therapeutically sufficient duration. In some embodiments, an agent is administered to a subject at a rate of 140-150 μg/kg/hour for a therapeutically sufficient duration. In some embodiments, an agent is administered to a subject at a rate of about 150 μg/kg/hour for a therapeutically sufficient duration. In some embodiments, an agent is administered to a subject at a rate of about 140 μg/kg/hour for a therapeutically sufficient duration. In some embodiments, an agent is administered to a subject at a rate of about 130 μg/kg/hour for a therapeutically sufficient duration. In some embodiments, an agent is administered to a subject at a rate of about 120 μg/kg/hour for a therapeutically sufficient duration. In some embodiments, an agent is administered to a subject at a rate of about 110 μg/kg/hour for a therapeutically sufficient duration. In some embodiments, an agent is administered to a subject at a rate of about 100 μg/kg/hour for a therapeutically sufficient duration. In some embodiments, an agent is administered to a subject at a rate of about 90 μg/kg/hour for a therapeutically sufficient duration.

In some embodiments, a therapeutically sufficient duration is at least 1 day. In some embodiments, a therapeutically sufficient duration is at least 2 days. In some embodiments, a therapeutically sufficient duration is at least 3 days. In some embodiments, a therapeutically sufficient duration is at least 4 days. In some embodiments, a therapeutically sufficient duration is at least 5 days. In some embodiments, a therapeutically sufficient duration is at least 6 days. In some embodiments, a therapeutically sufficient duration is at least 7 days. In some embodiments, a therapeutically sufficient duration is at least 8 days. In some embodiments, a therapeutically sufficient duration is at least 9 days. In some embodiments, a therapeutically sufficient duration is about 144 hours. In some embodiments, a therapeutically sufficient duration is about 140 hours. In some embodiments, a therapeutically sufficient duration is at least 24 hours. In some embodiments, a therapeutically sufficient duration is at least 48 hours. In some embodiments, a therapeutically sufficient duration is at least 60 hours.

In some embodiments, treatment comprises a step-wise decreasing of rate of administration. In some embodiments, treatment comprises the steps:
1. The rate is decreased to about 120 μg/kg/h for one hour;
2. The rate is further decreased to about 90 μg/kg/h for one hour;
3. The rate is further decreased to about 60 μg/kg/h for one hour; and
4. The rate is further decreased to about 30 μg/kg/h for one hour.

In some embodiments, the treatment comprise a decreasing of rate of administration. In some embodiments, the rate of administration is decreased from 70 μg/kg/h to 35 μg/kg/h.

In some embodiments, an agent is administered to a subject that is being or has been treated with mechanical ventilation. In some embodiments, administration of an agent (e.g. Compound 1 or Compound 2) continues throughout a subject's treatment with mechanical ventilation. In some embodiments, administration of an agent (e.g. Compound 1 or Compound 2) continues after a subject has ended treatment with mechanical ventilation. In some embodiments, the patient is intubated.

In some embodiments, Compound 1 or Compound 2 is administered to a subject who is receiving or has received treatment with a sedative. In some embodiments, a sedative is propofol or a benzodiazepine.

In some embodiments, the present disclosure includes administering to a subject in need thereof Compound 1 or Compound 2 in an amount sufficient to increase oxygen saturation in blood. In some embodiments, oxygen saturation in blood is measured using pulse oximetry.

In some embodiments, the present disclosure contemplates a method of treating a respiratory disease or condition, or a symptom of a respiratory disease or condition in a patient, wherein the patient is also experiencing a cytokine storm (also known as cytokine release syndrome). In some embodiments a method of treating a respiratory disease or condition, or a symptom of a respiratory disease or condition in a patient, wherein the patient is also experiencing a cytokine storm comprises the step of administering to the patient Compound 1 or Compound 2 or a pharmaceutically acceptable salt or formulation thereof. In some embodiments, a symptom of a cytokine storm is lung inflammation. In some embodiments, a patient undergoing a cytokine storm has acute respiratory distress syndrome (ARDS). In some embodiments, a symptom of a cytokine storm is multi-organ failure. In some embodiments, a patient undergoing treatment as described herein has elevated blood levels of cytokines. In some embodiments, the patient has elevated blood levels of IL-6, IL-1 and/or TNF-α. In some embodiments administration of an agent results in a decrease in blood levels of cytokines, e.g., IL-6, IL-1 and/or TNF-α. In some embodiments, a patient being treated as described herein is under 70 years of age; in other embodiments, the patient is 70 years of age or older.

In some embodiments, the present disclosure contemplates a method of treating a cytokine storm in a patient. In some embodiments a method of treating a cytokine storm comprising the step of administering to the patient Compound 1 or Compound 2 or a pharmaceutically acceptable salt or formulation thereof. In some embodiments, a symptom of a cytokine storm is lung inflammation. In some embodiments, a patient undergoing a cytokine storm has acute respiratory distress syndrome (ARDS). In some embodiments, a symptom of a cytokine storm is multi-organ failure. In some embodiments, a patient undergoing treatment as described herein has elevated blood levels of cytokines. In some embodiments, the patient has elevated blood levels of IL-6, IL-1 and/or TNF-α. In some embodiments administration of an agent results in a decrease in blood levels of cytokines, e.g., IL-6, IL-1 and/or TNF-α. In some embodiments, a patient being treated as described herein is under 70 years of age; in other embodiments, the patient is 70 years of age or older.

Infections

Another aspect of the disclosure contemplates, among other things, treatment of a subject who has an infection. The present disclosure contemplates, among other things, treatment of a subject who has a disease associated with an infection. In some embodiments, an infection is a viral infection or a bacterial infection or both. In some embodiments, an infection is a viral infection. In some embodiments, an infection is a bacterial infection.

In some embodiments, a viral infection is an infection of a virus selected from the group consisting of a coronavirus, an influenza virus, human rhinovirus, a human parainfluenza virus, human metapneumovirus and a hantavirus. In some embodiments, a virus is a coronavirus. In some embodiments, a coronavirus is selected from the group consisting of SARS-CoV, SARS-CoV-2, and MERS-CoV.

In some embodiments, a bacterial infection is an infection of a bacteria selected from the group consisting of Streptococcus pneumoniae, Chlamydia pneumoniae, Staphylococcus aureus, Pseudomonas aeruginosa, and Haemophilus influenzae. In some embodiments, Staphylococcus aureus is methicillin-resistant Staphylococcus aureus.

Respiratory Condition

In some embodiments, a subject with a respiratory condition suffers from respiratory distress. In some embodiments, respiratory distress includes acute respiratory distress.

In some embodiments a subject with a respiratory condition may exhibit one or more symptoms selected from the group consisting of airway hyper-responsiveness, inflammation of lung tissue, lung hypersensitivity, and inflammation-related pulmonary pain.

In some embodiments, a subject with a respiratory condition may exhibit inflammation of lung tissue. In some embodiments, inflammation of lung tissue is bronchitis or bronchiectasis. In some embodiments, inflammation of lung tissue is pneumonia. In some embodiments, pneumonia is ventilator-associated pneumonia or hospital-acquired pneumonia. In some embodiments, pneumonia is ventilator-associated pneumonia.

In some embodiments, administration of an agent to a subject exhibiting symptoms of a respiratory condition, results in reduction of the severity of respiratory distress in a subject with a respiratory condition or retard or slow the progression of respiratory distress in a subject with a respiratory condition.

In some embodiments, administration of an agent to a subject exhibiting symptoms of a respiratory condition, results in reduction of the severity of airway hyper-responsiveness in a subject with a disease associated with a coronavirus or retard or slow the progression of airway hyper-responsiveness in a subject with a respiratory condition.

In some embodiments, administration of an agent to a subject exhibiting symptoms of a respiratory condition, results in reduction of the severity of inflammation of lung tissue in a subject with a respiratory condition or retard or slow the progression of inflammation of lung tissue in a subject with a respiratory condition. In some embodiments, administration of an agent to a subject exhibiting symptoms of a respiratory condition, results in reduction of the severity of pneumonia in a subject with a respiratory condition or retard or slow the progression of pneumonia in a subject with a respiratory condition.

In some embodiments, administration of an agent to a subject exhibiting symptoms of a respiratory condition, results in reduction of the severity of lung hypersensitivity in a subject with a respiratory condition or retard or slow the progression of lung hypersensitivity in a subject with a respiratory condition.

In some embodiments, administration of an agent to a subject exhibiting symptoms of a respiratory condition, results in reduction of the severity of inflammation-related pulmonary pain in a subject with a respiratory condition or retard or slow the progression of inflammation-related pulmonary pain in a subject with a respiratory condition.

In some embodiments, administration of an agent to a subject exhibiting symptoms of a respiratory condition results in an improvement in one or more endpoints as described herein, e.g., as described in the Examples.

In some embodiments, a subject with a respiratory condition is undergoing or has undergone treatment for an infection, fibrosis, a fibrotic episode, chronic obstructive pulmonary disease, Sarcoidosis (or pulmonary sarcoidosis) or asthma/asthma-related inflammation.

In some embodiments, a subject exhibits symptoms of and/or has been diagnosed with asthma. In some embodiments, a subject is or has undergone an asthmatic attack.

In some embodiments, a subject is undergoing or has undergone treatment for fibrosis or a fibrotic episode. In some embodiments, the fibrosis is cystic fibrosis.

Another aspect of the present disclosure contemplates, among other things, a method of treating a subject who has or is being treated for a disease or condition selected from the group consisting of cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, pulmonary sarcoidosis, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington, spinocerebellar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, or Sjogren's disease.

Diseases Associated with a Coronavirus

Also described herein are methods of treating a subject wherein the subject exhibits one or more symptoms of a disease associated with a coronavirus and/or has been diagnosed with a disease associated with a coronavirus, comprising administering to said subject an agent selected from the group consisting of Compound 1 or Compound 2, or a pharmaceutically acceptable salt thereof. In some embodiments, the present disclosure contemplates a method of treating a subject comprising administering to said subject an agent selected from the group consisting of Compound 1 or Compound 2, or a pharmaceutically acceptable salt thereof, wherein the subject has a disease associated with a coronavirus.

In some embodiments, a disease associated with a coronavirus is selected from the group consisting of coronavirus disease 2019 (COVID-19), severe acute respiratory syndrome (SARS) and Middle East respiratory syndrome (MERS). In some embodiments, a disease associated with a coronavirus is selected from the group consisting of COVID-19. In some embodiments, a coronavirus is selected from a group consisting of SARS-CoV-1, SARS-CoV-2, and 2012-nCoV. In some embodiments, a coronavirus is SARS-CoV-2.

In some embodiments, administration of Compound 1 or Compound 2, or a pharmaceutically acceptable salt thereof, to a subject exhibiting symptoms of a disease associated with a coronavirus, may result in the reduction of the severity of one or more symptoms of a disease associated with a coronavirus or retard or slow the progression of one or more symptoms of a disease associated with a coronavirus.

In some embodiments, a subject with a disease associated with a coronavirus subject suffers from respiratory distress. In some embodiments, the respiratory distress is acute respiratory distress syndrome. In some embodiments, the subject exhibits a symptom selected from the group consisting of airway hyper-responsiveness, inflammation of lung tissue, lung hypersensitivity, and inflammation-related pulmonary pain. In some embodiments, the inflammation of lung tissue is bronchitis or bronchiectasis. In some embodiments, the inflammation of lung tissue is pneumonia. In some embodiments, the pneumonia is ventilator-associated pneumonia or hospital-acquired pneumonia.

In some embodiments, a subject with a disease associated with a coronavirus has been or is being treated with mechanical ventilation or oxygen. In some embodiments, a subject with a disease associated with a coronavirus has been or is being treated with mechanical ventilation. In some embodiments, the subject has or has been diagnosed with acute respiratory distress syndrome (ARDS).

In one aspect, the present disclosure contemplates a method of treating a subject (e.g., human subject) having a disease associated with coronavirus, comprising administering a first dose, e.g., a load dose of Compound 1 or Compound 2, e.g., to a subject under general anesthesia; administering a second dose, e.g., maintenance dose of Compound 1 or Compound 2, which is lower than said first dose; and administering a third dose, e.g., a downward taper dose of Compound 1 or Compound 2, said Compound 1 or Compound 2 dose being sufficient to treat said subject (e.g., human subject). In some embodiments, the maintenance dose comprises Compound 1. In some embodiments, the maintenance dose comprises Compound 2.

In one aspect, the present disclosure contemplates a method of treating a subject (e.g., human subject) having a disease associated with coronavirus, comprising administering a primary dose of an agent selected from the group consisting of Compound 1 or Compound 2, or a pharmaceutically acceptable salt thereof; and administering a subsequent dose of the agent that is lower than the first dose, e.g., a downward taper dose, said doses being sufficient to treat said subject (e.g., human subject).

In some embodiments, the primary dose has one or more of the characteristics of a first dose or load dose as described herein. In some embodiments, the primary dose has one or more of the characteristics of a second dose or maintenance dose as described herein. In some embodiments, the subsequent dose has one or more of the characteristics of a third dose or downward taper dose as described herein.

Symptoms of a Disease Associated with a Coronavirus

In some embodiments, a subject with a disease associated with a coronavirus suffers from respiratory distress. In some embodiments, respiratory distress includes acute respiratory distress. In some embodiments a subject with a disease associated with a coronavirus may exhibit one or more symptoms selected from the group consisting of airway hyper-responsiveness, inflammation of lung tissue, lung hypersensitivity, and inflammation-related pulmonary pain.

In some embodiments a subject with a disease associated with a coronavirus may exhibit inflammation of lung tissue. In some embodiments, inflammation of lung tissue is bronchitis. In some embodiments, inflammation of lung tissue is pneumonia. In some embodiments, pneumonia is ventilator-associated pneumonia or hospital-acquired pneumonia. In some embodiments, pneumonia is ventilator-associated pneumonia.

In some embodiments, administration of Compound 1 or Compound 2 to a subject exhibiting symptoms of a disease associated with a coronavirus, results in reduction of the severity of respiratory distress in a subject with a disease associated with a coronavirus or retard or slow the progression of respiratory distress in a subject with a disease associated with a coronavirus.

In some embodiments, administration of Compound 1 or Compound 2 to a subject exhibiting symptoms of a respiratory condition, results in an improvement in one or more endpoints as described herein, e.g., as described in the Examples.

In some embodiments, administration of Compound 1 or Compound 2 to a subject exhibiting symptoms of a disease associated with a coronavirus, results in reduction of the severity of airway hyper-responsiveness in a subject with a disease associated with a coronavirus or retard or slow the progression of airway hyper-responsiveness in a subject with a disease associated with a coronavirus.

In some embodiments, administration of Compound 1 or Compound 2 to a subject exhibiting symptoms of a disease associated with a coronavirus, results in reduction of the severity of inflammation of lung tissue in a subject with a disease associated with a coronavirus or retard or slow the progression of inflammation of lung tissue in a subject with a disease associated with a coronavirus. In some embodiments, administration of Compound 1 or Compound 2 to a subject exhibiting symptoms of a disease associated with a coronavirus, results in reduction of the severity of pneumonia in a subject with a disease associated with a coronavirus or retard or slow the progression of pneumonia in a subject with a disease associated with a coronavirus.

In some embodiments, administration of Compound 1 or Compound 2 to a subject exhibiting symptoms of a disease associated with a coronavirus results in reduction of the severity of lung hypersensitivity in a subject with a disease associated with a coronavirus or retard or slow the progression of lung hypersensitivity in a subject with a disease associated with a coronavirus.

In some embodiments, administration of Compound 1 or Compound 2 to a subject exhibiting symptoms of a disease associated with a coronavirus, results in reduction of the severity of inflammation-related pulmonary pain in a subject with a disease associated with a coronavirus or retard or slow the progression of inflammation-related pulmonary pain in a subject with a disease associated with a coronavirus.

Additional Therapeutics

The present disclosure contemplates, among other things administration of an agent to a subject has been previously administered a second agent selected from the group consisting of a bronchial muscle/airway relaxant, an antiviral, oxygen, an antibody, and an antibacterial. In some embodiments a second agent is administered to a subject prior to administration of an agent as described herein, the second agent is selected from the group consisting of a bronchial muscle/airway relaxant, an antiviral, oxygen, an antibody, and an antibacterial. In some embodiments, an agent as described herein is co-administered with to a subject with a second agent selected from a bronchial muscle/airway relaxant, an antiviral, oxygen, and an antibacterial.

In some embodiments, the antiviral is selected from the group consisting of remdesivir, kaletra, lopinavir, and ritonavir. In some embodiments, the antibody is sarilumab or tocilizumab. In some embodiments, the antibacterial is azithromycin.

### EXAMPLES

### Example 1—A Study of Brexanolone for Acute Respiratory Distress Syndrome Due to Covid-19 Using 150 Mcg/Kg/h Infusion

Primary Objective
To evaluate the effect of brexanolone on the duration of ventilator support in participants with acute respiratory distress syndrome (ARDS) due to COVID-19. Like Compound 1 and Compound 2, brexanolone is a GABA_{A} PAM.

Secondary Objective
To evaluate the safety of brexanolone in participants on ventilator support for ARDS due to COVID-19.

Other Objectives
To evaluate the effect of brexanolone on intensity of ventilator support in participants with ARDS due to COVID-19.
To evaluate the effect of brexanolone on pulmonary outcome measures.

Primary Endpoint
Time to extubation success (defined as >48 hours without need for reintubation or noninvasive ventilation).

Secondary Endpoint
Incidence of serious adverse events.

Other Endpoints
Change in oxygen saturation index (OSI) from baseline through the end of the infusion.
Change in oxygenation index (OI) from baseline through the end of the infusion.
Number of days until improvement in Berlin criteria defined ratio of partial pressure of arterial oxygen (PaO₂) to percentage of inspired oxygen (FiO₂) (PF ratio) severity category.
All-cause mortality.
Duration of stay in an intensive care unit (ICU) or equivalent setting Number of days of mechanical ventilation.
Pulmonary arterial pressure (where available).
Proportion of participants who progress to extracorporeal membrane oxygenation (ECMO).
Proportion of participants who require tracheostomy.

Study Description

The study will enroll approximately 50 participants with ARDS and SARS-CoV-2 infection who are currently intubated and receiving mechanical ventilation as part of standard of care, or who are on an immediate clinical plan to receive such intervention. These participants will receive brexanolone (also known as allopregnanolone) and will comprise the index case cohort and will be referred to as index participants.

In addition, case-controls with SARS-CoV-2 infection will be enrolled, matched 1:1 to index participants, based on age, sex, study site, and ARDS severity at time of diagnosis. These participants will not receive brexanolone and will comprise the case-control cohort and will be referred to as case-control participants. Case-control participants may be identified retrospectively via chart review, or they may be patients currently at the healthcare site who do not otherwise qualify for participation in the index case cohort (e.g., have been on ventilation for greater than 48 hours).

Index participants will receive a continuous IV infusion of brexanolone for 6 days (144 hours). For participants not on a ventilator at the time of screening, dosing should be initiated only after intubation and mechanical ventilation are in place and stabilized. Brexanolone will be administered at a dose of 150 mcg/kg/h; during the last 4 hours of the infusion a taper will be employed in the following manner: 120 mcg/kg/h for 1 h, 90 mcg/kg/h for 1 h, 60 mcg/kg/h for 1 h, 30 mcg/kg/h for 1 h. The taper should be initiated after 140 hours of infusion.

Postdose follow-up assessments will be conducted as summarized in Table 2; limited assessments will be collected by phone if the participant has already been discharged. All participants will have measures of pulmonary function assessed throughout the study.

Unless the clinical condition of the participant dictates otherwise, the ventilation guidelines for ARDS should be followed (Howell, 2018), as summarized here:
Target tidal volume of 4-8 mL/kg predicted body weight
Plateau pressure of <30 cm H₂O
Modest PEEP (peak end-expiratory pressure) values
Optional prone positioning for 12 hours per 24-hour period

Participant Inclusion Criteria

Each eligible participant must:
1. Be confirmed positive for SARS-CoV-2 infection as determined by polymerase chain reaction (PCR) or other commercial or public health assay at screening, or presumed positive with confirmation expected within 14 days of screening.
2. Be aged 2 years or older.
3. Be able to provide written informed consent, signed by participant or by proxy (legally acceptable representative), or enrolled under International Conference on Harmonisation (ICH) E6(R2) 4.8.15 emergency use provisions as deemed necessary by the investigator (for participants≥18 years of age).
4. Have a diagnosis of ARDS as confirmed with Berlin criteria.
5. Be intubated and receiving (or for case-control participants, have received) mechanical ventilation. Note that index participants must have initiated mechanical ventilation within 48 hours prior to screening, or have an immediate clinical plan for such intervention at time of screening.
6. Index participants must be likely to survive, in the opinion of the investigator, for at least 72 hours from the time of screening. Case-control participants must have survived for at least 72 hours from the time mechanical ventilation was initiated.

Participant Exclusion Criteria

Each eligible participant must not:
1. Be pregnant, based on self-report, report by family member, or pregnancy test if available.
2. Be in fulminant hepatic failure.
3. Have end stage renal disease.
4. Be participating in any other clinical trial of an experimental treatment with the same or similar primary endpoint.
5. Have a known allergy to progesterone, allopregnanolone, or any excipients in the brexanolone injection.

Additional Medications and/or Supplements

Participants should receive standard of care for ARDS, and all concomitant medications administered from the time of informed consent through the end of the study should be recorded on the appropriate CRF.

Brexanolone may potentiate the sedative effects of drugs used for ventilator support such as propofol and midazolam; the doses of these drugs should be titrated to the desired level of sedation during the infusion of brexanolone.

Efficacy Assessments

The intensity of ventilator support required to maintain adequate oxygenation will be assessed using the following endpoints, which are derived from inspired oxygen concentration and mean airway pressure using the following equations:

Oxygen Saturation Index (OSI) is (FiO₂×MAP×100)/SpO₂

Oxygen Index (OI) is (FiO₂/PaO₂)×MAP×100

PF ratio is PaO₂/FiO₂

Oxygen saturation (SpO₂) is the percentage of oxygen binding sites on hemoglobin that are bound by oxygen and will be assessed via pulse oximetry.

Mean airway pressure (MAP) refers to the mean pressure applied during positive-pressure ventilation. This metric will be read from the mechanical ventilator or will be calculated by the site.

Fraction of inspired oxygen (FiO₂) is the percentage of oxygen in the air mixture that is delivered to the subject. This metric will be read from the mechanical ventilator.

For participants with indwelling arterial catheter, partial pressure of oxygen (PaO₂) will be recorded. PaO₂ is a measurement of oxygen pressure in arterial blood, which reflects how well oxygen is able to move from the lungs to the blood.

Pulmonary arterial pressure is the direct or indirect measurement of blood pressure in the pulmonary artery. This will be measured only in participants with indwelling right heart catheters or by echocardiography when performed as standard of care.

PF ratio severity will be based on the PaO₂/FiO₂ cutoffs established by the Berlin Criteria, where mild=200 mmHg to ≤300 mmHg, moderate=100 mgHg to ≤200 mmHg, and severe=≤100 mmHg.

TABLE 2
Schedule of Assessments

| | Study Visit | | | | | | | | | |
|---|---|---|---|---|---|---|---|---|---|---|
| | | | | | | | | | V9 | |
| | V1 | | | | | | | V8 | V8 + 7 d | |
| | ≤48 h | | V3 | V4 | V5 | V6 | V7 | 145 h | (±1 d)¹ | V10 |
| | prior to | V2 | 25 h to | 48 h to | 73 h to | 97 h to | 121 h to | to | and/or | V9 + 14 d |
| | V2 | 0-24 h | 48 h | 72 h | 96 h | 120 h | 144 h | 168 h | ET | (±3 d) |
| Informed Consent | X | | | | | | | | | |
| Inclusion/Exclusion | X | | | | | | | | | |
| Demographics ² | X | | | | | | | | | |
| Medical History | X | | | | | | | | | |
| Body Weight/Height | I | | | | | | | | | |
| Berlin Criteria at time of | X | | | | | | | | | |
| ARDS diagnosis | | | | | | | | | | |
| Vital Signs ³ | I | | | | | | | | | |
| 12-Lead ECG ³ | I | | | | | | | | | |
| Hematology/ | I | | | | | | | | | |
| Chemistry ³ | | | | | | | | | | |
| Pregnancy test (as applicable) ⁴ | I | | | | | | | | | |
| Oxygen Saturation (%, SpO₂) ⁵ | X | X | X | X | X | X | X | X | X⁶ | |
| Mean Airway Pressure (MAP) ⁵ | X | X | X | X | X | X | X | X | X⁶ | |
| Fraction of Inspired Oxygen | X | X | X | X | X | X | X | X | X⁶ | |
| (FiO₂) ⁵ | | | | | | | | | | |
| Partial Pressure of Oxygen | X | X | X | X | X | X | X | X | X⁶ | |
| (PaO₂) ^{5, 7} | | | | | | | | | | |
| Pulmonary arterial pressure | X | X | | | | | | | X⁶ | |
| (if available) | | | | | | | | | | |
| Prone Positioning ⁸ | | X | X | X | X | X | X | X | X⁶ | |
| Administer Study Drug | | I⁹ | | | | | | | | |
| Data Collection at Discharge ¹⁰ | | | | | | | | | X⁶ | X |

| | |
|---|---|
| Serious Adverse Events | X |
| Concomitant Medications | X |

ABBREVIATIONS: ARDS = acute respiratory distress syndrome;
ECG = electrocardiogram;
ECMO = extracorporeal membrane oxygenation;
ET = early termination;
FiO₂ = fraction of inspired oxygen;
I = index participants only;
ICU = intensive care unit;
MAP = mean airway pressure;
PaO₂ = partial pressure of oxygen;
PF Ratio = PaO₂/FiO₂;
SpO₂ = oxygen saturation;
V = visit.
¹Follow-up by phone if participant has been discharged from inpatient care
² For case-control participants, demographic data will be limited to age and sex.
³ At screening, vital signs, ECG and chemistry/hematology may be recoded from assessments performed as part of standard of care on the day of screening, if available for index participants only. After screening, vital signs, ECG, and chemistry/hematology will be collected per standard of care and recorded only if pertinent for an SAE. Vital signs include supine systolic and diastolic blood pressure, heart rate, temperature, and respiratory rate (if not already on mechanical ventilation). ECG parameters include HR, PR, QRS, and QT interval plus brief descriptive text if the trace morphology is abnormal. Chemistry and hematology analytes include renal panel (glucose, calcium, phosphorus, blood urea nitrogen, creatinine, sodium, potassium, chloride, bicarbonate), hepatic panel (albumin, alanine aminotransferase, aspartate aminotransferase, total bilirubin, direct bilirubin, indirect bilirubin, alkaline phosphatase, total protein, lactate dehydrogenase, gamma glutamyl transferase), red blood cell count, hemoglobin, hematocrit, white blood cell count with differential, platelet count.
⁴ Urine or serum pregnancy test for female participant of childbearing potential, as needed. If a pregnancy test was done as part of standard of care, the result from that test may be documented.
⁵ Required at baseline and at the end of the infusion (for index participants); otherwise document as data are available.
⁶If still inpatient.
⁷ For participants with indwelling arterial catheter.
⁸ Document whether or not at least 12 hours of prone positioning was conducted each day during the period of intubation.
⁹Initiate brexanolone infusion at Hour 0. 4-hour taper to be initiated at Hour 140.
¹⁰ Data Collection at Discharge should include documentation of the date and time of the following, as applicable: discontinuation of extubation and mechanical ventilation; extubation failure requiring reintubation and re-initiation of mechanical ventilation; discharge from the ICU or equivalent. Also document whether or not the participant progressed to ECMO and/or tracheostomy and if so, the date and time of the associated procedure (ie, ECMO or tracheostomy). Also document whether or not the participant died, and if so, the date and cause of death.

### Example 2—A Study of Brexanolone for Acute Respiratory Distress Syndrome Due to Covid-19 Using 70 Mcg/Kg/h Infusion

Primary Objective
To evaluate the effect of brexanolone in participants on ventilator support for ARDS due to COVID-19. Like Compound 1 and Compound 2, brexanolone is a GABA_{A} PAM.

Secondary Objective
To evaluate the safety of brexanolone in participants on ventilator support for ARDS due to COVID-19.

Other Objectives
To evaluate the effect of brexanolone on pulmonary outcome measures in participants with ARDS due to COVID-19;
To evaluate the potential sedative-sparing effect of brexanolone in participants with ARDS due to COVID-19;
To evaluate the effect of brexanolone on cytokines and inflammatory markers in participants with ARDS due to COVID-19;
To assess the plasma pharmacokinetic (PK) profile of brexanolone in in participants with ARDS due to COVID-19.

Primary Endpoint
Proportion of participants alive and free of respiratory failure at Day 28.

Secondary Endpoint
Incidence of treatment-emergent AEs.

Other Endpoints
Time to successful extubation (defined as >48 hours without need for reintubation or noninvasive ventilation);
Change in oxygen saturation index (OSI) from baseline through the end of the infusion;
Change in oxygen index (OI) from baseline through the end of the infusion;
Change in PaO₂ to fraction of inspired oxygen (FiO₂) (PF ratio) from baseline through the end of the infusion;
All-cause mortality through Day 28;
Duration of stay in an ICU or equivalent setting;
Pulmonary arterial pressure (where available);
Change in hourly dose of midazolam or propofol from baseline through the end of infusion;
Changes from baseline in cytokines and inflammatory markers;
Change from baseline in vital signs, oxygen saturation, clinical laboratory parameters and 12-lead ECG;
PK parameters and exposure estimates derived from plasma concentration of brexanolone as assessed via population PK methods.

Study Description

This is a randomized, double-blind, placebo-controlled study designed to evaluate treatment with brexanolone (also known as allopregnanolone) in approximately 100 participants with ARDS due to COVID-19. Patients with ARDS and SARS-CoV-2 infection and who are currently intubated and receiving mechanical ventilation as part of standard of care or who are on an immediate clinical plan to receive such intervention will be eligible for screening. All participants must have mechanical ventilation in place prior to randomization. Eligible participants will be stratified by age (<70 or ≥70 years) and randomized 1:1 within each stratum to receive either brexanolone plus standard of care or placebo plus standard of care.

Participants will receive continued standard of care in addition to a continuous IV infusion of brexanolone or placebo for 60 hours. The infusion must be initiated within 6 hours from the time of randomization. The blinded 60-hour infusion will be administered at a dose of 70 mcg/kg/h for 58 hours followed by a 2-hour taper at 35 mcg/kg/h. Brexanolone is a solution for injection wherein each mL of solution contains 5 mg of brexanolone, 250 mg of betadex sulfobutyl ether sodium (solubilizer), citric acid and sodium citrate (buffering agents), and water, optionally with HCl or NaOH to adjust pH. Brexanolone solution is sterile-filtered and aseptically filled into 20 mL clear glass single-use vials and stored under refrigerated conditions (2 to 8° C.). The infusion dose is calculated based on weight of the participant (kg).

Brexanolone may potentiate the sedative effects of co-administered anesthetics (e.g., propofol or midazolam). During infusion, if sedation levels are deeper than intended, the dose of sedative anesthetics should be titrated to the desired effect.

Dose adjustment is permitted in the event of unplanned sedation/somnolence when participant is not receiving any sedating agents and/or if participant experiences an intolerable AE determined by investigator to be related to brexanolone infusion. The dose may be adjusted down to 35 mcg/kg/h or the infusion may be stopped. If symptoms resolve, the dosing may resume either at the reduced dosage (35 mcg/kg/h) or at the original dosage (70 mcg/kg/h).

Assessments will be conducted as summarized in Table 3. All participants will have measures of pulmonary function assessed throughout the study.

Participant Inclusion Criteria

Each eligible participant must:
1. Be confirmed positive for SARS-CoV-2 infection as determined by polymerase chain reaction (PCR) at screening;
2. Be aged 18 years or older;
3. Be able to provide written informed consent, signed by participant or by proxy (legally acceptable representative);
4. Have a presumptive diagnosis of ARDS at screening and PaO₂/FiO₂ (PF ratio)<300 prior to randomization;
5. Be intubated and receiving mechanical ventilation prior to randomization. Note that participants must have initiated mechanical ventilation within 48 hours prior to screening, or have an immediate clinical plan for such intervention at time of screening;
6. Be likely to survive, in the opinion of the investigator, for at least 72 hours from the time of screening.

Participant Exclusion Criteria

Each eligible participant must not:
1. Be pregnant, based on a positive pregnancy test at screening.
2. Be in fulminant hepatic failure at screening.
3. Have end stage renal disease at screening.
4. Have a known allergy to progesterone, allopregnanolone, or any excipients in the brexanolone injection.

Additional Medications and/or Supplements

Participants should receive standard of care treatment for ARDS due to COVID-19, and any concomitant medication deemed medically necessary for the welfare of the participant may be given at the discretion of the investigator at any time during the study. All concomitant medications, including central nervous system (CNS) depressants, agents employed to sedate participants and drugs administered to treat or prevent ICU delirium, administered from the time of informed consent through the end of the study, should be recorded. Phenytoin or propofol, if administered, should be administered in a separate line or via central line/midline port separate from brexanolone.

Efficacy Assessments

The primary endpoint is the proportion of participants alive and free of respiratory failure at Day 28. Respiratory failure is defined based on resource utilization, requiring at least one of the following:
endotracheal intubation and mechanical ventilation;
oxygen delivered by high-flow nasal cannula (heated, humidified oxygen delivered via reinforced nasal cannula at flow rates>20 L/min with fraction of delivered oxygen≥0.5;
noninvasive positive pressure ventilation;
extracorporeal membrane oxygenation (ECMO).

Pulmonary function will be assessed using the following endpoints, which are derived from fraction of inspired oxygen (FiO₂) and mean airway pressure (MAP) using the following equations:

Oxygen Saturation Index (OSI) is (FiO₂×MAP×100)/SpO₂

Oxygen Index (OI) is (FiO₂/PaO₂)×MAP×100

PF ratio is PaO₂/FiO₂

All assessments will be recorded at the time points as summarized in the Schedule of Assessments (Table 3).

Oxygen saturation (SpO₂) is the percentage of oxygen binding sites on hemoglobin that are bound by oxygen and will be assessed via pulse oximetry.

MAP refers to the mean pressure applied during positive-pressure ventilation. This metric will be read from the mechanical ventilator or will be calculated by the site.

Fraction of inspired oxygen (FiO₂) is the percentage of oxygen in the air mixture that is delivered to the participant. If necessary, FiO₂ may be estimated at screening based on room air.

Partial pressure of oxygen (PaO₂), as assessed via arterial blood sampling, will be recorded. PaO₂ is a measurement of oxygen pressure in arterial blood, which reflects how well oxygen is able to move from the lungs to the blood.

Pulmonary arterial pressure is the direct or indirect measurement of blood pressure in the pulmonary artery. This will be measured only in participants with indwelling right heart catheters or by echocardiography when performed as standard of care.

Prone positioning is recommended in guidelines for the management of severe ARDS. The site will document whether prone positioning for at least 12 hours per day occurred during the period of mechanical ventilation, and if not, the reason will be documented.

As applicable, the following will be documented during the follow-up visit(s):
Dates and times of extubation and discontinuation of mechanical ventilation
   a. The duration of time on mechanical ventilation will be calculated
   b. The time to successful extubation will be calculated
Dates and times of reintubation and re-initiation of mechanical ventilation.
Whether or not the participant requires oxygen delivered by high-flow nasal cannula heated, humidified oxygen delivered via reinforced nasal cannula at flow rates>20 L/min with fraction of delivered oxygen≥0.5), and if so the date and time of initiation of oxygen delivery by high-flow nasal cannula.
Whether or not the participant requires non-invasive positive pressure ventilation and if so, the date and time of initiation of non-invasive positive pressure ventilation.
Whether or not the participant progressed to ECMO, and if so, the date and time of initiation and (as applicable) completion of ECMO.
Date and time of discharge from the ICU or equivalent.
   The duration of the stay in the ICU or equivalent will be calculated
Whether or not the participant progressed to tracheostomy, and if so, the date and time of the tracheostomy procedure.
Whether or not the participant died, and if so, the date and cause of death.

Blood samples will be collected at the time points indicated in Table 3 and will be analyzed for cytokines and inflammatory markers.

Pharmacokinetic parameters will be estimated via population PK modeling.

Pharmacokinetic blood samples will be collected and processed for analysis for concentrations of brexanolone. Plasma samples for PK analysis will be collected according to the sampling schedule outlined in Table 3. An unscheduled plasma sample for PK analysis will also be collected for any participant who experiences an adverse event of special interest (AESI) as soon as is feasible after the onset of the event. In the event of an AESI, an unscheduled PK sample will be collected as soon as is feasible after the onset of the event. Bioanalysis of plasma samples for the determination of brexanolone will be performed using a validated liquid chromatography-tandem mass spectrometry method.

Safety Assessments

All safety assessments will be conducted according to the Schedule of Assessments (Table 3). Abnormalities in vital signs, ECGs, and out of range values in laboratory test results will be interpreted by an investigator as (1) abnormal, not clinically significant or (2) abnormal, clinically significant.

TABLE 3
Schedule of Assessments

| | Study Procedure | | | | | | | | | | |
|---|---|---|---|---|---|---|---|---|---|---|---|
| | Screening | Treatment Period | | | | | Follow-Up Period | | | | |
| | Study Visit | | | | | | | | | | |
| | | | | | | | | | | V10 | V11 |
| | | | | | | | | | | Day 14 | Day 28 |
| | V1 | | | | | | | | | (V9 + 7 d | (V10 + |
| | ≤48 h | V2 | V3 | V4 | V5 | V6 | V7 | V8 | V9 | [±1 d]¹) | 14 d |
| | prior to | 0 to | 12 h to | 24 h to | 36 h to | 48 h to | 60 h to | Day | Day | and/or | [±3 d]¹/End |
| | V2 | 12 h | 24 h | 36 h | 48 h | 60 h | 72 h | 5 | 7 | ET | of Study |
| Informed Consent | X | | | | | | | | | | |
| Inclusion/Exclusion | X | | | | | | | | | | |
| Demographics | X | | | | | | | | | | |
| Medical History | X | | | | | | | | | | |
| Body Weight/Height | X | | | | | | | | | | |
| Vital Signs ² | X | X | X | X | X | X | X | X | X | X¹¹ | |
| 12-Lead ECG ³ | X | | | | | X | | | | X¹¹ | |
| Hematology/Chemistry ⁴ | X | | | | | X⁵ | | | | X¹¹ | |
| Blood samples for | X | | | X | | X | | | X | X¹¹ | X¹¹ |
| cytokines and | | | | | | | | | | | |
| inflammatory markers ⁶ | | | | | | | | | | | |
| PK samples ⁷ | | | X | | | X | | | | | |
| Urine or serum | X | | | | | | | | | | |
| pregnancy test for | | | | | | | | | | | |
| female participants | | | | | | | | | | | |
| Oxygen Saturation | X | X | X | X | X | X | X | X | X | X¹¹ | |
| (%, SpO2) ⁸ | | | | | | | | | | | |
| Mean Airway Pressure | | X | X | X | X | X | X | X | X | X¹¹ | |
| (MAP) ⁹ | | | | | | | | | | | |
| Fraction of Inspired | X | X | X | X | X | X | X | X | X | X¹¹ | |
| Oxygen (FiO₂) ¹⁰ | | | | | | | | | | | |
| Partial Pressure of | X | X | X | X | X | X | X | X | X | X¹¹ | |
| Oxygen (PaO₂) ¹⁰ | | | | | | | | | | | |
| Pulmonary arterial | X | X | X | X | X | X | X | X | X | X¹¹ | |
| pressure | | | | | | | | | | | |
| (if available) ¹⁰ | | | | | | | | | | | |
| Pulmonary arterial | X | X | X | X | X | X | X | X | X | X¹¹ | |
| pressure | | | | | | | | | | | |
| (if available) ¹⁰ | | | | | | | | | | | |
| Prone Positioning ¹² | | X | | | | | | | | X¹¹ | X¹¹ |
| CAM-ICU ¹³ | | X | | | | | | | | X¹¹ | X¹¹ |
| RASS ¹³ | | X | | | | | | | | X¹¹ | X¹¹ |
| Randomization ¹⁴ | X | | | | | | | | | | |
| Administer | | X¹⁵ | | | | | | | | | |
| Investigational | | | | | | | | | | | |
| Product | | | | | | | | | | | |
| Follow-Up Data | | | | | | | | | | X | X |
| Collection ¹⁶ | | | | | | | | | | | |

| | |
|---|---|
| Adverse Events | X |
| Concomitant | X |
| Medications ¹⁷ | |

ABBREVIATIONS: AESI = adverse event of special interest;
ARDS = acute respiratory distress syndrome;
CAM-ICU = Confusion Assessment Method for the intensive care unit;
CTCAE = Common Terminology Criteria for Adverse Events;
ECG = electrocardiogram;
ECMO = extracorporeal membrane oxygenation;
ET = early termination;
FiO₂ = fraction of inspired oxygen;
ICU = intensive care unit;
MAP = mean airway pressure;
PaO₂ = partial pressure of oxygen;
PF Ratio = PaO₂/FiO₂;
SpO₂ = oxygen saturation;
RASS = Richmond-Agitation Sedation Scale;
V = visit
¹Follow-up by phone if participant has been discharged from inpatient care
² Vital signs to be assessed once at screening, predose, then approximately every 12 hours through Visit 7, and once at Visits 8 and 9. Also to be obtained once at Visit 10 if still inpatient. Vital signs include systolic and diastolic blood pressure, heart rate, temperature, and respiratory rate (if not on mechanical ventilation). Systolic and diastolic blood pressure and heart rate are to be collected supine or prone. Respiratory rate and temperature are collected once in any position. Additionally, respiratory rate, heart rate, and blood pressure should be collected for any participant who experiences an adverse event of special interest as soon as is feasible after the onset of the event.
³ ECG parameters to be assessed once daily at any time of day at the indicated visits. The following ECG parameters will be calculated and recorded: HR, PR, QRS, QT, and QTcF interval plus brief descriptive text if the trace morphology is abnormal.
⁴ To be collected once daily at the indicated visits. On Visit 6, collect within 30 minutes prior to the end of the infusion.
⁵ Sample to be collected at the same time as the PK sample collection obtained at 60 h, within 30 minutes prior to the end of the infusion
⁶ To be collected at 24 h (±30 minutes), at 58 h within 30 minutes prior to initiating taper. Samples to be obtained once at Visit 9 in addition to once at Visit 10 and at Visit 11 if still inpatient. The samples obtained at 24 h and 58 h should be obtained at the same time as the PK sample collections. Samples may be obtained at any time at Screening and at Visit 9, 10 and 11.
⁷ PK blood draws are to be collected at 24 h (±30 minutes) and 48 h (±30 minutes). On Visit 6, two separate samples should be collected: at 58 h within 30 minutes prior to initiating taper, and at 60 h, within 30 minutes prior to the end of the taper. The PK samples obtained at 24 h and 58 h should be obtained at the same time as the biomarker samples. An unscheduled plasma sample for PK analysis should also be collected for any participant who experiences an adverse event of special interest as soon as is feasible after the onset of the event; the time of collection must be recorded.
⁸ To be collected once at screening, predose, then approximately every 12 hours through Visit 7, then once at Visit 8 and Visit 9. Also to be obtained once at Visit 10 if still inpatient. One of the assessments must be obtained at the end of the infusion (60 h ± 30 minutes). Where possible when scheduled at the same time point, assessments should be collected at the same time as MAP, PaO₂, and FiO₂ and pulmonary arterial pressure (if available). Additionally, SpO₂ should be collected with vital signs for any participant who experiences an adverse event of special interest as soon as is feasible after the onset of the event.
⁹ To be collected predose, then approximately every 12 hours through Visit 7, then once at Visit 8 and Visit 9. Also to be obtained once at Visit 10 if still inpatient. One of the assessments must be obtained at the end of the infusion (60 h ± 30 minutes). Where possible when scheduled at the same time point, assessments should be collected at the same time as PaO₂, FiO₂, SpO₂, and pulmonary arterial pressure (if available).
¹⁰ To be collected once at screening, predose, then approximately every 12 hours through Visit 7, then once at Visit 8 and Visit 9. Also to be collected once at Visit 10 if still inpatient. One of the assessments must be obtained at the end of the infusion (60 h ± 30 minutes). Where possible when scheduled at the same time point, assessments should be collected at the same time as MAP and SpO₂. Prior to placement and initiation of mechanical ventilation, FiO₂ may be estimated from room air at screening if necessary.
¹¹ If still inpatient.
¹² Document whether or not at least 12 hours of prone positioning was conducted each day during the period of mechanical ventilation.
¹³ RASS to be administered daily and within 2 hours after each extubation for all participants. Also to be administered each time the decision is made to stop the IP infusion or reduce the dose due to an AE. CAM-ICU to accompany RASS when RASS score ≥−3.
¹⁴ Not to occur prior to placement and initiation of mechanical ventilation.
¹⁵ Initiate infusion at Hour 0; a 2-hour taper to be initiated at Hour 58. Dose adjustments are permitted if a participant meets the criteria.
¹⁶ Information to be documented at Visit 10 includes: “dates and times of extubation and discontinuation of mechanical ventilation”, “dates and times of reintubation and re-initiation of mechanical ventilation”, “whether or not the participant requires oxygen delivered by high-flow nasal cannula (heated, humidified oxygen delivered via reinforced nasal cannula at flow rates >20L/min with fraction of delivered oxygen ≥0.5), and if so, the date and time of initiation of oxygen delivery by high-flow nasal cannula”, “whether or not the participant requires non-invasive positive pressure ventilation and if so, the date and time of initiation of non-invasive positive pressure ventilation”, “whether or not the participant progressed to ECMO, and if so, the date and time of initiation and (as applicable) completion of ECMO”, “date and time of discharge from the intensive care unit or equivalent”, “whether or not the participant progressed to tracheostomy, and if so, the date and time of the tracheostomy procedure”, “whether or not the participant died, and if so, the date and cause of death”. All information is to be updated as necessary at Visit 11.
¹⁷ All medications taken from the time of informed consent through the duration of the study will be recorded. Details such as the start/stop dates and times, dose, and indication will be recorded. In addition, any change to the dose(s), and date and time of each dosage change should be recorded for sedative drugs, CNS depressants, and drugs administered to treat or prevent ICU delirium.

### Example 3—An Exemplary Study of the Positive Allosteric Modulators of the GABA_{A} Receptor (GABA_{A} PAM), Compound 1 and Compound 2 in the Treatment of a Disease or Condition, or a Symptom of a Respiratory Disease or Condition in a Human Subject

Compound 1 or Compound 2 could be administered to a number of human subjects who are contemporaneously afflicted by a respiratory disease or condition such as respiratory distress (e.g. acute respiratory distress syndrome), to determine efficacy in the treatment of the disease. For example, Compound 1 or Compound 2 could be administered to half of the subjects, while the other half of the subjects would be administered a placebo, and the effects of the treatment on the symptom(s) of the disease or condition being studied could then be compared between the two groups. The present study would include guidelines to ensure the reliability of the results, such as inclusion/exclusion criteria, specific objectives, specific procedure, reliable data analysis, efficacy assessments, and safety assessments. The present study could be used to assess the effects of the administration of Compound 1 or Compound 2 on symptoms of respiratory distress, such as airway hyper-responsiveness, inflammation of lung tissue (such as bronchitis, bronchiectasis or pneumonia), lung hypersensitivity, or inflammation-related pulmonary pain. The present study could also be used to assess the effects of the administration of Compound 1 or Compound 2 on symptoms of respiratory distress in subjects who are undergoing or have undergone treatment for a viral infection (such as coronavirus (e.g. SARS-CoV, SARS-CoV-2, and MERS-CoV), an influenza virus, human rhinovirus, a human parainfluenza virus, human metapneumovirus and a hantavirus), a bacterial infection (such as Streptococcus pneumoniae, Chlamydia pneumoniae, Staphylococcus aureus (e.g. methicillin-resistant Staphylococcus aureus), Pseudomonas aeruginosa, and Haemophilus influenza), fibrosis (such as cystic fibrosis), a fibrotic episode, chronic obstructive pulmonary disease, Sarcoidosis (or pulmonary sarcoidosis) or asthma/asthma-related inflammation. The present study could be used to assess whether participants who would be administered Compound 1 or Compound 2 would be alive and free of respiratory failure at Day 28. Respiratory failure could defined based on resource utilization, requiring at least one of the following: endotracheal intubation and mechanical ventilation; endotracheal intubation and mechanical ventilation; oxygen delivered by high-flow nasal cannula (heated, humidified oxygen delivered via reinforced nasal cannula at flow rates>20 L/min with fraction of delivered oxygen≥0.5; noninvasive positive pressure ventilation; extracorporeal membrane oxygenation (ECMO).

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or all of the group members are present in, employed in, or otherwise relevant to a given product or process.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A method of treating a respiratory disease or condition, or a symptom of a respiratory disease or condition in a subject in a subject, comprising administering to said subject a therapeutically effective amount of a GABA_{A} PAM, or a pharmaceutically acceptable salt or crystalline form thereof, wherein the GABA_{A} PAM is selected from the group consisting of Compound 1 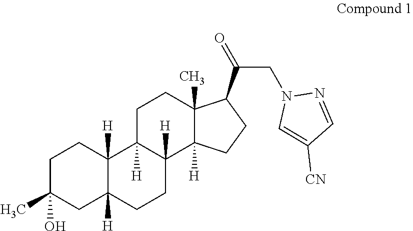
and Compound 2

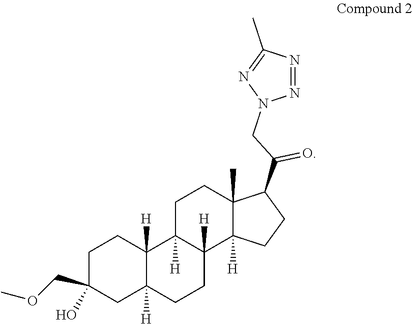

2. The method of claim 1, wherein the respiratory condition is respiratory distress.

3. The method of claim 1 or claim 2, wherein the respiratory condition is acute respiratory distress syndrome.

4. The method of any one of claims 1-3, wherein the subject exhibits a symptom of the respiratory condition, wherein the symptom is selected from the group consisting of airway hyper-responsiveness, inflammation of lung tissue, lung hypersensitivity, and inflammation-related pulmonary pain.

5. The method of claim 4, wherein the inflammation of lung tissue is bronchitis or bronchiectasis.

6. The method of claim 5, wherein the inflammation of lung tissue is pneumonia.

7. The method of claim 6, wherein the pneumonia is ventilator-associated pneumonia or hospital-acquired pneumonia.

8. The method of any one of claims 1-7, wherein the subject is undergoing or has undergone treatment for an infection, fibrosis, a fibrotic episode, chronic obstructive pulmonary disease, Sarcoidosis (or pulmonary sarcoidosis) or asthma/asthma-related inflammation.

9. The method of claim 8, wherein the subject is undergoing or has undergone treatment for an infection.

10. The method of claim 9, wherein the infection is a viral infection.

11. The method of claim 10, wherein the viral infection is an infection of a virus selected from the group consisting of a coronavirus, an influenza virus, human rhinovirus, a human parainfluenza virus, human metapneumovirus and a hantavirus.

12. The method of claim 11, wherein the virus is a coronavirus selected from the group consisting of SARS-CoV, SARS-CoV-2, and MERS-CoV.

13. The method of claim 12, wherein the coronavirus is SARS-CoV-2.

14. The method of any one of claims 1-13, wherein the subject has been or is being treated for a disease selected from SARS, COVID-19 or MERS.

15. The method of claim 14, wherein the disease is COVID-19.

16. The method of claim 9, wherein the infection is a bacterial infection.

17. The method of claim 16, wherein the bacterial infection is selected from the group consisting of Streptococcus pneumoniae, Chlamydia pneumoniae, Staphylococcus aureus, Pseudomonas aeruginosa, and Haemophilus influenzae.

18. The method of claim 17, wherein the Staphylococcus aureus is methicillin-resistant Staphylococcus aureus.

19. The method of claim 8, wherein the subject is undergoing or has undergone treatment for fibrosis or a fibrotic episode.

20. The method of claim 19, wherein the fibrosis is cystic fibrosis.

21. The method of any one of claims 1-20, further comprising administering to the subject one or more additional agents selected from a group consisting of a bronchial muscle/airway relaxant, an antiviral, oxygen, an antibody, and an antibacterial.

22. The method of claim 21, wherein the one or more additional agents are administered to the subject prior to, after, or concurrently with the GABA_{A} PAM.

23. The method of any of claims 21-22, wherein the antiviral agent is selected from the group consisting of remdesivir, kaletra, lopinavir, and ritonavir.

24. The method of any of claims 21-23, wherein the antibody is sarilumab or tocilizumab.

25. The method of any of claims 21-24, wherein the antibacterial is azithromycin.

26. The method of any one of claims 1-25, wherein the subject is being treated, or has been treated with an oxygen mask.

27. The method of any one of claims 1-26, wherein the subject is being treated, or has been treated with mechanical ventilation.

28. The method of claim 27, wherein the GABA_{A} PAM is administered to a subject until the subject is no longer being treated with mechanical ventilation.

29. The method of any one of claims 1-28, wherein the subject has been or is being treated for a disease or condition selected from the group consisting of cystic fibrosis, asthma, smoke induced COPD, chronic bronchitis, rhinosinusitis, constipation, pancreatitis, pancreatic insufficiency, male infertility caused by congenital bilateral absence of the vas deferens (CBAVD), mild pulmonary disease, pulmonary sarcoidosis, idiopathic pancreatitis, allergic bronchopulmonary aspergillosis (ABPA), liver disease, hereditary emphysema, hereditary hemochromatosis, coagulation-fibrinolysis deficiencies, such as protein C deficiency, Type 1 hereditary angioedema, lipid processing deficiencies, such as familial hypercholesterolemia, Type 1 chylomicronemia, abetalipoproteinemia, lysosomal storage diseases, such as I-cell disease/pseudo-Hurler, mucopolysaccharidoses, Sandhof/Tay-Sachs, Crigler-Najjar type II, polyendocrinopathy/hyperinsulemia, Diabetes mellitus, Laron dwarfism, myleoperoxidase deficiency, primary hypoparathyroidism, melanoma, glycanosis CDG type 1, congenital hyperthyroidism, osteogenesis imperfecta, hereditary hypofibrinogenemia, ACT deficiency, Diabetes insipidus (DI), neurophyseal DI, neprogenic DI, Charcot-Marie Tooth syndrome, Perlizaeus-Merzbacher disease, neurodegenerative diseases such as Alzheimer's disease, Parkinson's disease, amyotrophic lateral sclerosis, progressive supranuclear palsy, Pick's disease, several polyglutamine neurological disorders such as Huntington, spinocerebellar ataxia type I, spinal and bulbar muscular atrophy, dentatorubal pallidoluysian, and myotonic dystrophy, as well as spongiform encephalopathies, such as hereditary Creutzfeldt-Jakob disease (due to prion protein processing defect), Fabry disease, Straussler-Scheinker syndrome, COPD, dry-eye disease, and Sjogren's disease.

30. The method of any one of claims 1-29, wherein the GABA_{A} PAM is administered via inhalation, intravenously, intramuscularly, subcutaneously, or orally.

31. The method of claim 30, wherein the GABA_{A} PAM is administered orally.

32. The method of claim 30, wherein the GABA_{A} PAM is administered intravenously.

33. The method of claim 32, wherein the GABA_{A} PAM is administered by continuous intravenous infusion

34. The method of claim 33, wherein the GABA_{A} PAM is administered at a rate of 90-160 μg/kg per hour for a therapeutically effective duration.

35. The method of claim 34, wherein the GABA_{A} PAM is administered at a rate of 90-150 μg/kg/hour.

36. The method of claim 35, wherein the GABA_{A} PAM is administered at a rate of 130-150 μg/kg/hour.

37. The method of claim 36, wherein the GABA_{A} PAM is administered at a rate of 140-150 μg/kg/hour.

38. The method of claim 34, wherein the GABA_{A} PAM is administered at a rate of about 150 μg/kg/hour.

39. The method of claim 34, wherein the GABA_{A} PAM is administered at a rate of about 140 μg/kg/hour.

40. The method of claim 34, wherein the GABA_{A} PAM is administered at a rate of about 120 μg/kg/hour.

41. The method of claim 34, wherein the GABA_{A} PAM is administered at a rate of about 100 μg/kg/hour.

42. The method of claim 33, wherein the GABA_{A} PAM is administered at a rate of 10-100 μg/kg/hour, for a therapeutically effective duration.

43. The method of claim 42, wherein the GABA_{A} PAM is administered at a rate of 30-80 μg/kg/hour.

44. The method of claim 43, wherein the GABA_{A} PAM is administered at a rate of 35-70 μg/kg/hour.

45. The method of claim 42, wherein the GABA_{A} PAM is administered at a rate of 70 μg/kg/hour.

46. The method of claim 42, wherein the GABA_{A} PAM is administered at a rate of 35 μg/kg/hour.

47. The method of claim 42, wherein the GABA_{A} PAM is administered at a rate of 60-80 μg/kg/hour.

48. The method of any one of claims 34-47, wherein the therapeutically effective duration is at least 24 hours.

49. The method of claim 48, wherein the therapeutically effective duration is at least 48 hours.

50. The method of claim 49, wherein the therapeutically effective duration is at least 60 hours.

51. The method of claim 50, wherein the therapeutically effective duration is at least 3 days.

52. The method of claim 51, wherein the therapeutically effective duration is at least 4 days.

53. The method of claim 52, wherein the therapeutically effective duration is at least 5 days.

54. The method of claim 53, wherein the therapeutically effective duration is at least 6 days.

55. The method of claim 34, wherein the GABA_{A} PAM is administered at a rate of 150 μg/kg/h for about 140 hours.

56. The method of claim 58, further comprising the steps of:
a. decreasing the administration rate to about 120 μg/kg/h for one hour;
b. further decreasing the administration rate to about 90 μg/kg/h for one hour;
c. further decreasing the administration rate to about 60 μg/kg/h for one hour; and
d. further decreasing the administration rate to about 30 μg/kg/h for one hour.

57. The method of claim 42, wherein the GABA_{A} PAM is administered at a rate of 70 μg/kg/h for about 58 hours.

58. The method of claim 57, further comprising decreasing the rate of administration of the GABA_{A} PAM to about 35 μg/kg/h for about 2 hours.

59. The method of any one of claims 1-58, wherein treating a respiratory disease or condition in a subject comprises ameliorating one or more symptoms of the respiratory disease or condition.

60. The method of claim 59, comprising ameliorating one or more symptoms of acute respiratory distress syndrome in a subject.

61. The method of claim 59, comprising ameliorating one or more symptoms of COVID-19 in a subject.

62. The method of any of claims 59-61, wherein the symptom is selected from the group consisting of airway hyper-responsiveness, inflammation of lung tissue, lung hypersensitivity, and inflammation-related pulmonary pain.

63. The method of any one of claims 1-62, wherein the GABA_{A} PAM is administered to the subject in an amount sufficient to increase oxygen saturation in the blood of the subject.

64. The method of claim 63, wherein the oxygen saturation in the blood of the subject is measured using pulse oximetry.

65. The method of any one of claims 1-64, wherein the subject is also experiencing a cytokine storm (also known as cytokine release syndrome).

66. The method of claim 65, wherein the patient has an inflammation resulting from cytokine release syndrome.

67. The method of claim 66, wherein the inflammation is a lung inflammation.

68. The method of any one of claims 65-67, wherein the patient has acute respiratory distress syndrome.

69. The method of any one of claims 1-68, wherein the patient is intubated.

70. The method of any one of claims 1-69, wherein the patient is under 70 years of age.

71. The method of any one of claims 1-69, wherein the patient is 70 years of age or older.

72. The method of any one of claims 1-71, wherein the GABA_{A} PAM is Compound 1, wherein the Compound 1 is a free base.

73. The method of any one of claims 1-71, wherein the GABA_{A} PAM is a pharmaceutically acceptable salt of Compound 1.

74. The method of any one of claims 1-71, wherein the GABA_{A} PAM is Compound 2, wherein the Compound 2 is a free base.

75. The method of any one of claims 1-71, wherein the GABA_{A} PAM is a pharmaceutically acceptable salt of Compound 2.
